(19) United States
(12) Patent Application Publication
(10) Pub. No.: US 2023/0377691 A1
(43) Pub. Date: Nov. 23, 2023

(54) ESTIMATING PREDISPOSITION FOR DISEASE BASED ON CLASSIFICATION OF ARTIFICAL IMAGE OBJECTS CREATED FROM OMICS DATA

(71) Applicant: THE BOARD OF REGENTS OF THE UNIVERSITY OF TEXAS SYSTEM, Austin, TX (US)

(72) Inventor: Xiangning CHEN, Germantown, MD (US)

(21) Appl. No.: 18/231,390

(22) Filed: Aug. 8, 2023

Related U.S. Application Data
(63) Continuation of application No. 16/887,909, filed on May 29, 2020.
(60) Provisional application No. 62/855,762, filed on May 31, 2019.

(51) Int. Cl. G16B 40/00; C12Q 1/6827; G16B 30/00; G16B 20/00

(52) U.S. Cl. CPC G16B 40/00; C12Q 1/6827; G16B 30/00; G16B 20/00

(57) ABSTRACT

Methods and systems are provided that receive biological trait information of a subject and biological trait information of controls; and generate artificial image objects (AIOs) from the biological trait information, where each AIO is formed of an array of cells that are single unit addressable (e.g., x,y coordinates). Each cell of the AIO for the subject and the AIOs for the controls is accorded a specific graphic pixel signal corresponding to at least one data type of specific variant information of an assigned discrete unit of the biological trait information for that cell. The AIOs for the controls form a training set of artificial image objects, which are used to train an artificial intelligence (AI) algorithm for classifying AIOs. The trained AI algorithm is applied to the AIO for the subject to determine a probability that a particular biological trait is present in the subject.

### CROSS-REFERENCE TO RELATED APPLICATIONS

This application is a Continuation Application of U.S. patent application Ser. No. 16/887,909, filed May 29, 2020, which claims the benefit of U.S. Provisional Patent Application Ser. No. 62/855,762, filed May 31, 2019, which is hereby incorporated by reference in its entirety.

### BACKGROUND

Over the last 50 years, research has proven various links between genetic variations, gene expression, protein expression variations, and protein functions, to human disease. Human disease and susceptibility to human disease have also been linked to epigenetic variations, metabolomics variations, microbiomic variations, proteomics variations, and other “omics” molecular biology variations, where “omics” commonly refers to the study of various biological systems, such as proteomics, genomics, transcriptomics, microbiomics, metabolomics, glycomics, lipomics, and the like. These variations, individually and collectively, contribute to and influence the development of human characteristics and diseases, which are commonly referred to as phenotypes or traits in biomedical and genetic studies. To a complex trait, the contributing variants could be in the hundreds, thousands or more. For many complex human diseases, many risk variants have not been discovered. It remains a great challenge to not only discover and characterize these molecular biology variants, but also to use this large amount of variant information and data to improve disease identification, diagnosis, intervention, treatment, prognosis, and, ultimately, the mental and physical health of a human being.

Current approaches to utilize knowledge of molecular biology variants of the types mentioned above for disease risk assessment, diagnosis, and personalized treatment can be grouped into two general approaches. The first approach is to use variant data individually or group them into specific panels. For example, there are reports that multiple Genome-Wide Association Study (GWAS)-associated variants, such as single nucleotide variations (SNVs), are used as a panel to assess risks for diabetes (Wu et al, Scientific Reports, 7:43709, 2017: Go et al., J. Human Genetics, 61(12):1009-1012, 2016; Chatterjee et al., Nature Genetics, 45(4):400-405e3, 2013), cancers (Kuchenbaecker et al., J. Nat. Canc. Inst., 109(7):djw302, 2017; Wen et al., Breast Canc. Res., 18(1):124, 2016), heart disease (McNamara et al., Circulation: Card. Gen., 3:226-228, 2010; Harst et al., Circ. Res., 122(3).433-443, 2018; and Smith et al., PLoS Genet., 6(9):e1001094, 2010), and schizophrenia and bipolar disorder (Vassos et al., Biol. Psych., 81(6):470-477, 2017; Maier et al., Am. J. Human Genetics, 96(2):283-294, 2015; Maier et al., Nat. Commun., 91(1):989, 2018). To use these GWAS-associated variants effectively, researchers have applied a variety of procedures to select and evaluate individual variants or markers before incorporating them into a panel. The number of SNVs included in a panel are limited, varying from tens to hundreds. Due to the small effective sizes of individual SNVs and the limited number of SNVs included in a panel, the performances of most panels are not satisfactory.

The second approach to use such variant data is to calculate an aggregate value based on the effects of variants on the trait or disease of interest. The most common algorithm used for this purpose is referred to as a polygenic analysis, where the effects of individual SNVs on the trait are summed up and normalized by the number of SNVs included in the analysis. There are many reports of polygenic risk score (PRS) applications in disease association, diagnosis, treatment response, and prognosis. (See. Kuchenbaecker et al., 2017; Escott-Price et al., J. Neurol., 138(Pt. 12) 3673-3684, 2015; Domingue et al., PLoS One, 9(7) e101596, 2014; and Chen et al., J. Neurimmon. Pharmacol., 13(4):532-540, 2019). An important issue in polygenic analysis is to evaluate the optimal threshold to decide which SNVs should be included in the study. In most studies, PRSs are calculated at rather liberal P-value thresholds (P=0.01, 0.1, 0.5 or larger). At a substantially smaller P-value, such as P≤5×10⁻⁵, the performance of PRSs is usually unsatisfactory. Furthermore, due to the fact that the PRS is an aggregate score, SNVs with opposite effects lose their utility. These weaknesses, to some extent, limit the usefulness of this approach in most clinical settings.

Molecular biology variations, such as genetic variations, or protein expression variations, etc., are ubiquitous in human beings and differ from individual to individual. Many such variations lead to no perceived differences in susceptibility or predisposition to disease. Yet, these variations are the raw sources of evolution, and to a large extent, nonetheless determine various human traits that can lead to disease, including common diseases such as cardiovascular or heart diseases (such as, but not limited to, atherosclerotic vascular disease, myocardial infarction, heart failure, hypertrophic myocardiomyopathy, pericarditis, coronary artery disease, cardiomegaly, and the like), cancers, and mental disorders. It remains a great challenge to identify which variants are responsible for these disease traits and how best to utilize the volumes of variant data to address large-scale health care issues.

In recent years, biomedical research has produced a large amount of biological data that poses a great challenge to analyze for disease diagnosis, treatment, and prevention. These data include DNA sequencing data (genetic variations), genomic, and epigenomic data, protein functional assay data, metabolomics, microbiome, and other biomarker data. To date there exists no unified single methodology that is capable of transforming this large volume of genetic and other biomarker data into actionable information for diagnosis, treatment, and prevention of disease.

Thus, presented herein are Artificial Image Objects (AIOs) and their use in methods that quickly, accurately, and reproducibly identify the presence of desired traits in subjects in need thereof. Also provided are systems used for this purpose. These methods operate by transforming variant data into AIOs, arrangements of variant data as graphic pixel signals into two or more dimensions, and analyzing the pixel signals collectively using highly sophisticated, state-of-the-art Artificial Intelligence (AI), machine learning (ML), and artificial neural networks (ANN). These methods are used to build statistical models for disease risk assessment, disease diagnosis, treatment response and prognosis, and prediction models for other human behaviors and traits. In the methods and systems described herein, millions of genetic variants and other biological variant markers are analyzed by employing image processing and analytic algorithms. These method and systems therefore provide an efficient and effective tool for discovery of relationships between sets of genetic variants or other biomarkers and any biological trait of interest. The methods described herein are useful for disease diagnosis, risk assessment, treatment response, and trajectory, as well as prediction of human behaviors or mental disorders.

### SUMMARY

Disclosed herein are methods of classification of biological data for the purpose of identifying whether or not a subject of interest possesses the classified trait. Biological data include genetic variations and the like. For instance, biological data include genetic data, protein data, epigenomic data, microbiome data, proteome data, and the like. For instance, genetic data includes, for example, genetic data, such as copy number data, gene expression data, and/or single nucleotide variation (SNV) data.

Generally, the methods disclosed herein involve several steps. The steps generally comprise construction of one or more artificial image objects (AIO) comprising biological data followed by artificial intelligence (AI)-assisted analysis of the AIOs. The AI-assisted analysis involves learning which AIOs possess image-specific trait information and which do not. Based on this analysis, there follows the determination of whether a given AIO from a given subject possesses the trait of interest or not.

Thus, the methods disclosed herein include analysis of AIOs constructed from numerous different types of biological data. In one embodiment, the biological data is genetic data. In methods utilizing genetic data, the methods include steps such as obtaining a first set of genetic variants from a first subject, wherein the first subject is the subject for which determination of the presence of the trait is desired. Other steps include obtaining a second set of genetic variants obtained from a population of one or more second subjects. In this embodiment of the disclosed methods, the second subjects are control subjects, i.e., subjects for which the presence or absence of the desired trait is known. The second subjects therefore include subjects that possess the trait and subjects that do not possess the trait. The biological data information for the one or more second subjects is in one embodiment publicly-available. That is, the trait information is, in one embodiment, obtained from a public database of such information. In another embodiment, the trait information is obtained firsthand by performing assays on subjects to obtain trait data, such as genetic variant data and the like. In another embodiment, the trait data is proprietary or otherwise owned by a public or private entity and obtained through license or acquisition by other means. This information is included in the obtained genetic variant information. In this embodiment, the first set of genetic variants and the second set of genetic variants are of the same set of genetic variants, and the population of one or more second subjects comprises subjects possessing the genetic trait and subjects not possessing the genetic trait.

From these sets of variants, a first two-dimensional AIO is generated. In this embodiment the AIO is a genetic AIO The AIO is optionally two- or three-dimensional, or optionally more than three-dimensional. In other words, the AIO comprises several different types of biological data encoded into the AIO AIOs comprise a plurality of cells, wherein each cell in the AIO corresponds to a single genetic variant obtained from the first subject. Each cell is assigned a mutually distinguishable shading intensity or color and each of the mutually distinguishable shading intensities or colors corresponds to a specific genotype, for instance as represented by the homozygous/heterozygous symbology as AA, Aa, or aa, etc.

Thereafter, in this embodiment, a plurality of second two-dimensional genetic AIOs are generated, each comprising a plurality of cells as with the first AIO, wherein each one of the second genetic AIOs corresponds to one of the one or more second subjects, and wherein each cell in each of the second genetic AIOs is assigned to the same single genetic variant assigned for each corresponding cell in the first genetic AI. Each genotype is also assigned the same mutually distinguishable shading intensity or color as assigned in the first genetic AIO.

In addition to generating multiple AIOs from multiple sources of data, etc., an artificial intelligence (AI) algorithm is trained on the plurality of second genetic AIOs. In other words, the AIO information is inputted into the AI for processing by the AI program. Processing by the AI program results in indexing of the spatial relationships between each of the cells in each of the AIOs. Initially, the plurality of second genetic AIOs is processed by the AI in an AI training step such that the corresponding shading intensities of each the plurality of cells therein are distinguishing between AIOs with the genetic trait and AIOs without the genetic trait.

In this embodiment, after the AI has been trained on the AIOs of the second subjects, i.e., the control subjects, and is capable of distinguishing between a trait-containing AIO and a non-trait-containing AIO, then the AIO from the first subject is processed by the AI. From this step there is obtained from the AI analysis a determination whether the first genetic AIO possess the genetic trait or not, and thereby whether the first subject possess the genetic trait.

In another embodiment of the disclosed methods, the method includes the further step of selecting a specific subset of genetic variants from the first set of genetic variant data and the second set of genetic variant data. The selection process is based on any number of factors. In one embodiment, the selection is based on a genome-wide association study (GWAS) and/or linkage disequilibrium (LD) value. In this embodiment, the genetic AIOs are generated solely based on the sub-set of selected genetic variants.

In one embodiment of the disclosed methods, the step of generating the first genetic AIO comprises at least the following steps: (a) assigning a single selected genetic variant to each cell of the first genetic AIO such that each cell corresponds to a different genetic variant, (b) assigning a mutually distinguishable shading intensity and/or color to each genotype, and (c) assigning a shade and/or color to each cell of the first genetic AIO based on the assigned genetic variants and the genotypes of the first subject for these variants.

In yet another embodiment of the disclosed methods, the step of generating the plurality of second genetic AIOs comprises at least the following steps: (a) assigning the same selected genetic variants to the same cells of the plurality of second genetic AIOs, (b) assigning the same mutually distinguishable shading intensity and/or color to each genotype, and (c) shading and/or coloring each cell of the plurality of second genetic AIOs based on the assigned genetic variants and the genotypes of the second subject for these variants.

Other embodiments of the disclosed methods, as mentioned above, involve different types of genetic variant information. For example, in one embodiment, the genetic variant data comprises one or more copy number variants (CNV) and/or one or more single nucleotide variations (SNV), and/or one or more gene expression levels.

In certain embodiments of the disclosed methods, the AI algorithm is a machine learning (ML) algorithm, such as, for instance, an artificial neural network (ANN). In other embodiments of the disclosed methods, the ANN is one or more of a convolutional neural network (CNN), a deep learning neural network (DNN), a deep, highly nonlinear neural network (NNN), a developmental network (DN), a long short-term memory network (LSTM), a recurrent neural network (RNN), a deep belief network (DBN), large memory storage and retrieval neural network (LAMSTAR), deep stacking network (DSN), spike-and-slab restricted Boltzmann machine network (ssRBM), and a multilayer kernel machine network (MKM). In one embodiment, the artificial neural network is a convolutional neural network (CNN), and the CNN comprises at least one convolutional layer. In another embodiment, the AI algorithm includes an optimizer program, optionally wherein the optimizer program is a tensorflow optimizer program.

The methods disclosed herein are therefore generally directed to determining, through AI-assisted classification processes, whether or not a subject possesses a certain biological trait. In some embodiments, the trait is a genetic trait. In some embodiments, the genetic trait is a predisposition to one or more mental illnesses, such as, for instance, a neurodevelopmental disorder, schizophrenia, bipolar disorder, anxiety disorder, trauma related disorder, dissociative disorder, somatic symptom disorder, eating disorder, sleeping disorder, impulsive/disruptive/conduct disorder, addictive disorder, neurocognitive disorder, or a personality disorder. In such embodiments, the methods optionally comprise the additional active step of prescribing counseling to the subject and/or administering a pharmaceutically active agent to the subject, who is determined to possess the trait in question, that treats the mental illness if the genetic trait is present.

In another such embodiment, the genetic trait is susceptibility to cancer, such as, for instance, a carcinoma, sarcoma, myeloma, leukemia, or lymphoma. In such embodiments, where the subject is determined to possess the biological trait of interest, and wherein the trait is a predisposition or susceptibility to cancer, the methods in such embodiments optionally include a further active step of administering a pharmaceutically active agent to the subject that treats the cancer if the trait is present.

The methods described herein are directed to identification of the presence of one or more biological traits in a subject. In such methods, the subject is a human, alpaca, cattle, bison, camel, deer, donkey, elk, goat, rat, mouse, horse, llama, mule, rabbit, pig, sheep, buffalo, monkey, ape, yak, dog, cat, chicken, fish, duck, goose, or hamster.

Additional methods are described herein that are similar to those mentioned above, but instead of utilizing genetic data (epigenomics, SNVs, CNVs, etc.), they utilize protein-based data, such as protein expression, post-translational modifications, and other protein functional information Thus, in another embodiment, disclosed are methods of classification for detection of a trait in a subject from one or more AIOs representing protein function and/or protein expression data. Such methods comprise the steps mentioned above, including, for instance, obtaining a first set of protein function and/or protein expression data from a first subject, i.e., the subject for whom the presence of the biological trait is desired, as well as obtaining a second set of protein function and/or protein expression data obtained from a population of one or more second subjects, i.e., “control” subjects that are either known to possess the trait or not possess the trait (information that is included in the data). These data are generally publicly available or otherwise obtainable through known empirical methodologies.

In such methods, the first set of gene function and/or gene expression data and the second set of gene function and/or gene expression data are of the same set of gene function and/or gene expression data, and the population of one or more second subjects includes both subjects possessing the trait and subjects not possessing the trait. In another step, the method calls for generating a first two-dimensional expression AIO comprising a plurality of cells, wherein each cell in the protein AIO corresponds to a single gene function and/or gene expression data obtained from the first subject. In such AIOs, as in the above methods, each cell is assigned a mutually distinguishable shading intensity or color, and each of the mutually distinguishable shading intensities or colors corresponds to the level of gene function and/or gene expression amount of the first subject. In another step of the method, a plurality of second two-dimensional expression AIOs are generated, each comprising a plurality of cells, wherein each one of the second expression AIOs corresponds to one of the one or more second subjects, and wherein each cell in each of the second AIOs is assigned to the same single gene function and/or gene expression data assigned for each corresponding cell in the first expression AIO. In such methods, each level of gene function/gene expression is assigned the same mutually distinguishable shading intensity or color as assigned in the first expression AIO based on the level of gene function and/or gene expression amount of the one or more second subjects.

In some embodiments, the gene expression and/or gene expression data is transcription variant data. In such embodiments, various transcription variants are known, such as one or more of: a) alternative splicing variants, selected from exon skipping variants, intron retention variants, alternative 5′ splicing variants, alternative 3′ splicing variants, alternative first exon variants, and/or alternative last exon variants, and b) allele-specific alternative splicing variants.

As with the above-described methods based on genetic information, in the present expression-based embodiments, the methods include training an AI algorithm on the plurality of second expression AIOs, thereby indexing spatial relationships between each of the cells in each of the plurality of second expression AIOs and corresponding shading intensities of each the plurality of cells therein, such that the AI is capable of distinguishing between expression AIOs with the trait and expression AIOs without the trait. Finally, in such methods, the first expression AIO is analyzed by the AI, after which a determination if whether the first expression AIO possesses the trait is obtained from the AI, and thereby whether the subject possesses the trait.

As in the above-described methods directed to the utilization of genetic information, in the present embodiment directed to the use of gene expression based data, there are optionally additional steps, wherein generating the first expression AIO comprises, for example, assigning a single gene function and/or gene expression to each cell of the first expression AIO such that each cell corresponds to a different gene function and/or gene expression data, and assigning a mutually distinguishable shading intensity and/or color to each gene function and/or gene expression. Additionally, in this embodiment, a shade and/or color is assigned to each cell of the first expression AIO based on the assigned gene function and/or gene expression data and the level of gene function and/or gene expression obtained from the first subject.

Likewise, in another embodiment of such methods, generating the plurality of second expression AIOs comprises several steps, such as assigning the same selected gene function and/or gene expression data points to the same cells of the plurality of second expression AIOs, as well as assigning the same mutually distinguishable shading intensity and/or color to each level of gene function and/or gene expression. Finally, in such embodiments, each cell of the plurality of second expression AIOs is shaded and/or colored based on the assigned gene function and/or gene expression data and the level of gene function and/or gene expression for the one or more second subjects.

In some embodiments of such methods, the gene function and/or gene expression data comprises one or more gene expression level, and/or one or more gene function data point.

In certain embodiments that take into consideration also variances in protein-level information, the method further optionally comprises obtaining two sets of protein function and/or protein expression data, one set of data from the first subject and a second set of data from a population of one or more second subjects, wherein the first set of protein function and/or protein expression data and the second set of protein function and/or protein expression data are of the same set of protein function and/or protein expression data, wherein the population of one or more second subjects comprises subjects possessing the genetic trait and subjects not possessing the genetic trait, and wherein the AIO is generated with the sets of protein function and/or protein expression data, the AI is trained with the AIO comprising these additional data, and the determination of whether the first AIO possesses the trait is based on the AIO generated with the protein function and/or protein expression data. In such embodiments, the protein function and/or protein expression data comprises one or more protein expression levels. In some embodiments, the protein function and/or protein expression data comprises one or more protein function data points. In some embodiments, the protein function and/or protein expression data comprises one or more one or more post-translational modification variant data points.

For example, the post-translational modification variants are optionally selected from one or more of ubiquitination, alkylation, phosphorylation, disulfide bond formation, carbonylation, carboxylation, acylation, acetylation, glycosylation, prenylation, amidation, hydroxylation, adenylylation, and carbamylation.

In some embodiments of the expression-based AIO methods, the AI algorithm is a machine learning algorithm, such as, but not limited to, an artificial neural network (ANN). In some of these embodiments utilizing an ANN, the ANN is one or more of a convolutional neural network (CNN), a deep learning neural network (DNN), a deep, highly nonlinear neural network (NNN), a developmental network (DN), a long short-term memory network (LSTM), a recurrent neural network (RNN), a deep belief network (DBN), large memory storage and retrieval neural network (LAMSTAR), deep stacking network (DSN), spike-and-slab restricted Boltzmann machine network (ssRBM), and a multilayer kernel machine network (MKM). In embodiments of the method that include utilization of a CNN artificial neural network, the CNN optionally comprises at least one convolutional layer. Additionally, in some embodiments, the methods include the utilization of an AI program that further comprises an optimizer program, optionally the optimizer is a tensorflow optimizer program.

In some embodiments of the described methods, the trait in question is a disposition towards one or more mental illnesses. In such embodiments, the one or more mental illnesses is one or more of a neurodevelopmental disorder, schizophrenia, bipolar disorder, anxiety disorder, trauma related disorder, dissociative disorder, somatic symptom disorder, eating disorder, sleeping disorder, impulsive/disruptive/conduct disorder, addictive disorder, neurocognitive disorder, or a personality disorder. In such embodiments directed to traits that are characteristic of mental disorders, the method optionally includes the additional active step of prescribing counseling to the subject and/or administering a pharmaceutically active agent to the subject that treats the mental illness if the trait is present. Alternatively, in some embodiments, the trait is a susceptibility or predisposition to cancer or a cancer subtype. In such embodiments, the cancer is a carcinoma, sarcoma, myeloma, leukemia, or lymphoma in such embodiments directed to cancer, the methods optionally comprise an additional active step of administering a pharmaceutically active agent to the subject that treats the cancer if the trait is present.

The subjects in the disclosed methods are human, alpaca, cattle, bison, camel, deer, donkey, elk, goat, rat, mouse, horse, llama, mule, rabbit, pig, sheep, buffalo, monkey, ape, yak, dog, cat, chicken, fish, duck, goose, or hamster, for instance.

As described above, in some embodiments of the described methods, the AIO is a two-dimensional MO. However, this is merely one embodiment. In other embodiments, the method employs a three-dimensional AIOs. Additional dimensions are optionally added to the AIO depending on the number of data sets to be included in the analysis by the AI. For example, in embodiments in which there are at least three dimensions to the AIO, the third dimension comprises variants obtained from the first subject and/or the one or more second subjects at different time points. Optionally, in this embodiment, the third dimension is encoded into the AIO by assigning different colors for each different time point. Thus, in some embodiments, the AIO comprises at least three dimensions, wherein each of the three dimensions corresponds to data selected from at least the following types of data: genetic variant data, gene expression data, proteomic data, epigenomic data, metabolomic data, and microbiome data.

In another embodiment, the different dimensions encoded by the AIO are based on data sets obtained at different times. That is, for example, in one embodiment the data is obtained at time=0 for all data sets, and then another set of data of all types included in the AIO are obtained at a second time, time=0+x. These two different data sets are included then in the AIO and analyzed by the AI as above. The term “x” in this embodiment is any quantity from hours, days, months, to years.

In another embodiment, two or more different data types each form an AIO, and two or more AIOs from the same subjects are used for training with AI algorithms to determine whether the subject possesses the trait of interest or does not possess the trait.

### BRIEF DESCRIPTION OF THE DRAWINGS/FIGURES

FIG. 1 provides a visual flow chart of certain steps of the disclosed methods, including: 1) obtaining genetic variants: 2) generating an Artificial Image Object (AIO) by recoding and arranging genetic variant data into a digital image; and 3) training an Artificial Intelligence (AI) algorithm on the AIOs to classify AIOs.

FIGS. 2A and 2B provide a visual flow chart depicting the recoding and rearrangement of genotype data into Artificial Image Objects (AIOs). FIG. 2A shows an exemplary process in gray-scale Each SNV Genotype (aa, aA, and AA) is assigned a distinct shade or intensity of the gray-scale (left panel). Each SNV is also assigned to a distinct cell within the AIO and the gray-scale value converted into a numerical value (0, 154, and 254, middle panel) The AIO is generated based on these inputs (right two panels). In this example, the black pixels represent AA genotypes, gray pixels represent heterozygous, i.e., Aa genotypes, and white pixels represent aa genotypes, at the specified AIO cell addresses. FIG. 2B shows an exemplary process similar to that shown in FIG. 2A except in a 3-color scheme Each color is assigned to a subset of the SNV genotype data and forms a color layer in the AIO (left panels). For each color, genotypes (aa, aA, and AA) are each assigned a distinct shade or intensity of the assigned color that are converted into numerical values (0, 154, and 254, middle two panels). The overlay of the three color forms a 3-color AIO (right two panels, colors in the image are represented as various shades of gray). In the 3-color AIO, the black and white cell signals indicate that all three layers have the same AA and as genotypes at the specified cell addresses. As an illustrative scenario when using colors, pure red, blue, and green signals can indicate that only one layer has signals at these addresses. Then, yellow signals can be the result of a combination of red and green layers, the magenta signals can be from a combination of red and blue layers, and cyan signals can be from blue and green combination.

FIGS. 3A, 3B, 3C, and 3D provide AIOs and performance data, corresponding to Example 3, of binary classification with GW AS-selected SNVs. FIG. 3A is a representative 3-color AIO for a schizophrenia patient where 120,000 SNVs (200/200-3) are incorporated into the AIO FIG. 3B is a representative 3-color AIO for a healthy subject where the same 120,000 SNVs are incorporated into the AIO. FIGS. 3C and 3D show 2-D plots of data obtained from a typical training nm of the neural network model to classify the schizophrenia patients and normal controls (colors are represented as various shades of gray). FIG. 3C shows the training and validation accuracy in terms of accuracy vs. epoch while FIG. 31) shows the AUC in terms of true positive rate vs. false positive rate.

FIGS. 4A, 4B, 4C, 4D, and 4E show the performance of a multi-category classification corresponding to Example 4 where AIOs generated from 33,075 SNVs (105×105×3) were used to classify lung cancer subtypes from normal samples. FIG. 4A is a representative 3-color AIO for the normal samples to be classified. FIGS. 4B and 4C are AIOs for the adenocarcinoma and squamous cell lung cancer subtypes respectively. FIGS. 4D and 4E show a typical training run of the neural network model used to classify the 3 groups of samples where 4D shows the training accuracy in terms of accuracy vs. epoch, while 4E shows the AUC in terms of a 2-D plot of true positive rate vs. false positive rate.

FIGS. 5A, 5B, 5C, and 5D provide AIOs and performance data corresponding to Example 5, a binary classification of breast cancer subtypes (Ki67⁺ and Ki67⁻) using gene expression data. FIG. 5A is a representative 3-color AIO for a Ki67⁺ patient incorporating 16,875 genes (75×75×3) to generate the AIO. FIG. 5B is a representative 3-color AIO for a Ki67⁻ subject incorporating the same 16,875 genes to generate the AIO. FIGS. 5C and 5D are plots of data obtained from a typical training run of the neural network model showing the performance measurement values (accuracy in FIG. 5C, and AUC in FIG. 5D).

FIGS. 6A, 6B, and 6C provide a depiction of performance data corresponding to Example 6, a multi-category classification of breast cancer subtypes (PAM50) using gene expression data. FIG. 6A is a representative AIO made from gene expression data (75×75×3 genes). FIG. 6B is a plot of training accuracy of the model and FIG. 6C is a ROC curve of the training run.

### DETAILED DESCRIPTION

### Definitions

The following terms are used throughout the disclosure, the definitions of which are provided herein to assist in understanding one or more aspects of the disclosure, including the claims. The definitions include various examples that fall within the scope of a term and that may be used for implementation and are not intended to be limiting.

Unless defined otherwise, all technical and scientific terms used herein have the same meaning as commonly understood by those of ordinary skill in the art in which this disclosure resides. Although any methods and materials similar or equivalent to those described herein are useful in the practice or testing of the presently disclosed compositions and methods, in some cases preferred or exemplary methods and materials are described herein.

As used herein, the word “exemplary” means “serving as an example, instance, or illustration.” Any embodiment described herein as “exemplary” is not necessarily to be construed as preferred or advantageous over other embodiments. Furthermore, there is no intention to be bound by any theory presented in the preceding background or the following detailed description.

It is to be noted that the term “a” or “an” entity refers to one or more of that entity; for example, “a binding molecule,” is understood to represent one or more binding molecules. As such, the terms “a” (or “an”), “one or more,” and “at least one” can be used interchangeably herein.

Furthermore, “and/or” where used herein is to be taken as specific disclosure of each of the two specified features or components with or without the other. Thus, the term “and/or” as used in a phrase such as “A and/or B” herein is intended to include “A and B,” “A or B,” “A” (alone), and “B” (alone). Likewise, the term “and/or” as used in a phrase such as “A, B, and/or C” is intended to encompass each of the following embodiments: A, B, and C; A, B, or C; A or C, A or B; B or C; A and C; A and B; B and C; A (alone); B (alone); and C (alone).

As used herein, the term “about” or “approximately” refers to a variation of 10% from the indicated values (e.g., 50%, 45%, 40%, etc.), or in case of a range of values, means a 10% variation from both the lower and upper limits of such ranges. For instance, “about 50%” refers to a range of between 45% and 55%.

Unless defined otherwise, technical and scientific terms used herein have the same meaning as commonly understood by one of ordinary skill in the art to which this disclosure is related. For example, the Concise Dictionary of Biomedicine and Molecular Biology, Juo, Pei-Show, 2nd ed., 2002, CRC Press; The Dictionary of Cell and Molecular Biology, 3rd ed., 1999, Academic Press; and the Oxford Dictionary Of Biochemistry And Molecular Biology, Revised, 2000, Oxford University Press, provide one of skill with a general dictionary of many of the terms used in this disclosure.

Units, prefixes, and symbols are denoted in their Systéme International de Unites (SI) accepted form. Numeric ranges are inclusive of the numbers defining the range. Unless otherwise indicated, amino acid sequences are written left to right in amino to carboxy orientation. DNA sequences are written left to right in the 5′ to 3′ direction. The headings provided herein are not limitations of the various aspects or aspects of the disclosure, which can be had by reference to the specification as a whole. Accordingly, the terms defined immediately below are more fully defined by reference to the specification in its entirety.

As used herein, the term “bipolar disorder” refers to a disease, also known as manic-depressive illness, that is a brain disorder that causes unusual shifts in mood, energy, activity levels, and the ability to carry out day-to-day tasks for any subject suffering therefrom. Bipolar disorder can be broken down into four main types, including type I, type II, cyclothymic, and other/unspecified bipolar and related disorders. Subjects with bipolar disorder experience periods of unusually intense emotion, changes in sleep patterns and activity levels, and unusual behaviors called “mood episodes,” which are drastically different from the moods and behaviors that are typical for a subject of the same age. Bipolar disorder is defined by the Diagnostic and Statistical Manual of Mental Disorders (DSM-5) as six different sub-types each of which is diagnosed based on a specific set of criteria. (See, Substance Abuse and Mental Health Services Administration. DSM-5 Changes: Implications for Child Serious Emotional Disturbance [Internet]. Rockville (Md.). Substance Abuse and Mental Health Services Administration (US); 2016 June. Table 12, DSM-IV to DSM-5 Bipolar I Disorder Comparison). Furthermore, various GWAS studies centered around diagnosed schizophrenia have been published. (See, for instance, Stahl et al, bioRxiv 173062; doi: doi.org/10.1101/173062; The Wellcome Trust Case Control Consonium, Nature, 447(7145):661-678, 2007; and Hou et al., Hum. Mol. Genet., 25(15).338³-3394, 2016).

The term “psychotic disorder” or “mental disorder” as used herein refers to a disorder in which psychosis is a recognized symptom, this includes neuropsychiatric (psychotic depression and other psychotic episodes) and neurodevelopmental disorders (especially Autistic spectrum disorders), neurodegenerative disorders, depression, mania, and in particular, schizophrenic disorders (paranoid, catatonic, disorganized, undifferentiated, and residual schizophrenia) and bipolar disorders.

As used herein, the term “depression” (also called major depressive disorder, or clinical depression) is a psychiatric mood disorder that can be categorized into various diseases including persistent depressive disorder, perinatal depression, psychotic depression, seasonal affective disorder, and bipolar disorder. Depression often results in a loss of social function, reduced quality of life and increased mortality. The World Health Organization estimates that roughly 322 million people suffer from clinical depression. (World Health Organization (WHO) (2017); “Depression and Other Common Mental Disorders: Global Health Estimates,” Geneva World Health Organization). This disorder can occur from infancy to old age, with women being affected more often than men Depression can have many causes that range from genetic, over psychological factors (negative self-concept, pessimism, anxiety, and compulsive states, etc.) to psychological trauma. (See, Leubner et al., Front. Psychol., 8.1109, 2017). Depression is associated with a chronic, low-grade inflammatory response and activation of cell-mediated immunity, as well as activation of the compensatory anti-inflammatory reflex system. (See, Berk et al., BMC Med., 11:200, 2013). Evidence suggests that clinical depression can be accompanied by increased oxidative and nitrosative stress (O&NS) and autoimmune responses directed against O&NS modified neoepitopes (Id.)

The term “schizophrenia” as used herein is defined by the DSM 5 as a spectrum disorder having five key symptoms, including delusions, hallucinations, disorganized speech, disorganized or catatonic behavior, and negative symptoms. DSM 5 also defines other related conditions on the spectrum including, for instance, schizoaffective disorder and delusional disorder (See, American Psychiatric Association, Diagnostic and Statistical Manual of Mental Disorders, Fifth Edition, American Psychiatric Publishing, Washington, D.C., 2013 Pages 99-105). Furthermore, various GWAS studies centered around diagnosed schizophrenia have been published. (See, for instance, Pardinas et al., Nat. Genet., 50(3):381-389, 2018: and Schizophrenia Working Group of the Psychiatric Genomics Consortium, Nature, 511:421-427, 2014).

As used herein, the term “administered,” or “administration,” or “to administer,” means administration of a pharmaceutically active pharmaceutical ingredient (API) or composition thereof, the composition is administered to the subject, or contacting the subject with the API. The API is administered by any of the known ways in which to administer such APIs, for example as a topical application, oral dosage, subcutaneous injection, intramuscular injection, intraperitoneal injection, intravenous injection, intrathecal dosage, and/or intradermal injection, and the like.

Terms such as “treating” or “treatment” or “to treat” or “alleviating” or “to alleviate” refer to therapeutic measures that cure, slow down, lessen symptoms of, and/or halt or slow the progression of an existing diagnosed pathologic condition or disorder. Terms such as “prevent,” “prevention.” “avoid,” “deterrence” and the like refer to prophylactic or preventative measures that prevent the development of an undiagnosed targeted pathologic condition or disorder.

By “subject” or “individual” or “animal” or “patient” or “mammal,” is meant any subject, particularly a mammalian subject, for whom diagnosis, prognosis, or therapy is desired. Mammalian subjects include humans, domestic animals, farm animals, and zoo, sports, or pet animals such as dogs, cats, guinea pigs, rabbits, rats, mice, horses, swine, cows, bears, and so on. For instance, subjects include, but are not limited to, human, alpaca, cattle, bison, camel, deer, donkey, elk, goat, rat, mouse, horse, llama, mule, rabbit, pig, sheep, buffalo, monkey, ape, yak, dog, cat, chicken, fish, duck, goose, and hamster. In some instances, the subject is a plant or tree, such as a common agricultural plant crop or tree crop, some non-limiting examples of which are corn, soybean, cotton, rice (maize), wheat, potato, apple, orange, coffee, peanut, rapeseed, onion, bean, cacao, beet, and Cannabis, etc.

A “trait,” as used herein, means one or more characteristics or attributes of an organism that are expressed by genes and/or influenced by the environment. When expressed by genes, the term is referred to as a “genetic trait.” A genetic trait is any genetically-determined characteristic of an organism. Traits include, for example, physical attributes of an organism, behavioral characteristics, and susceptibility to, or predisposition for, disease. Traits refer to a feature, physical or chemical, of an organism. A trait is a distinct variant of a phenotypic characteristic of an organism that may be either inherited or determined environmentally, or some combination thereof.

A “genetic variant,” as used herein, refers to a variance in a specific piece of genetic information. Thus, genetic variants include single nucleotide variations (SNVs), differences in gene expression, copy number variants (CNVs), differences in epigenomics, and the like.

By “genotype,” as used herein, is meant the genetic constitution of an individual organism. An individual organism's genotype is its complete heritable genetic identity, i.e., its unique genome as revealed by personal genome sequencing “Genotype” also refers to a particular genetic variant or set of genetic variants carried by an individual. In contrast, a “phenotype” is a description of the individual's actual physical characteristics, which is influenced by genotypes, epigenetic factors, and non-inherited environmental factors in which the individual lives, i.e., the genotype of an individual contributes to its phenotype. An individual's genetic makeup is often described by a particular gene of interest and a combination of alleles that the individual carries, i.e., homozygous or heterozygous. Genotypes are often symbolized in English by two letters that are a combination of upper case and lower case, such as AA, Aa, and aa, where “A” stands for one allele and “a” stands for the other allele. That is, for a diploid organism such as a human, two alleles make three different and distinguishable genotypes.

A “singe nucleotide variation” as used herein refers to a difference of identity of a single nucleotide at a single position within a single genome, or a missing nucleotide, often called an insertion or deletion (“indel”) at a single position within a single genome. These differences at single positions in a genome between individuals can be found in coding or non-coding sequences of genes. Further, such SN Vs can be synonymous or nonsynonymous, i.e., a change that alters the identity of the amino acid sequence of the encoded protein, or a change that does not alter the identity of the amino acid sequence of the encoded protein, respectively. According to the U.S. National Library of Medicine. Genetics Home Reference, there are more than 100 million known SNVs across the human population. For example, a specific human genome will, on average, differ from the reference human genome at between 4 and 5 million different specific positions within the genome. Some nonsynonymous SNVs result in substitution of one amino acid for another (missense mutation), or even the creation of a new stop codon (nonsense mutation). Synonymous SNVs have been linked to changes in expression of genes that can ultimately lead to disease. The National Center for Biotechnology Information (NCBI) publishes a database of SNVs (dbSNP) that includes over 893 million human SNVs (build 151, 2017) Other publicly-available databases of SNVs include the OMIM database, Kaviar, SNPedia, dbSAP, the International HapMap Project, and the like. (See also, ensembl org, the European Bioinformatics Institute, for a listing of currently available variant databases). Currently there are over 500,000 variations known to be associated with a phenotype or clinical disease, according to ClinVar, from the U S National Center for Biotechnology Information.

The term “copy number variation” (CNV) as used herein, means a genetic alteration that is a type of structural variant involving alterations in the number of copies of specific regions of DNA in an individual's genome. Such regions of CNV DNA are either deleted or duplicated, in some cases duplicated multiple times in a single individual genome (See, Thapar et al., J. Am. Acad. Child.Adolesc. Psychiatrv, 52(8):772-774, 2013). The U. S. National Cancer Institute defines CNV as a genetic trait involving the number of copies of a particular gene present in the genome of an individual. Genetic variants, including insertions, deletions, and duplications of segments of DNA, are also collectively referred to herein as copy number variants. To date there are over 500,000 CNVs that have been reported and described in the human genome. (See, for instance, the Database of Genomic Variants, at dgv.tcag.ca). While most CNVs are not directly linked to disease, there are several reported instances of CNV abnormalities contributing to disease because they occur in critical developmental genes, such as Huntington's disease. Public data bases are available, including the Wellcome Trust Sanger Institute DECIPHER CNV database that associates known CNVs with known clinical conditions. (See also, Daar et al., Nature Reviews Genetics, 7:414, 2006). It is known, for instance, that somatic-derived copy number variants are frequent in neuron cells in the human brain.

As used herein, the terms “susceptibility” or “predisposition” means the quality or state of being susceptible to something, i.e., lack of ability to resist an extraneous agent, such as a drug or pathogen. Susceptibility means the degree of the likelihood of being liable to being influenced or harmed by a condition, i.e., an inherent biological weakness towards succumbing to a health condition, such as a mental abnormality or cancer. Likewise, the term “predisposition” as used herein means that a subject has not yet developed the disease or health abnormality or other diagnostic criteria but, nevertheless, has a likelihood to develop the disease or abnormality within a defined time window in the future (predictive window) with a certain likelihood That is, the term “predisposition” as used herein means that a subject does not currently present with the disease or disorder, but is liable to be affected by the disorder in time.

The term “diagnosis” as used herein encompasses identification, confirmation, and/or characterization of a disease or disorder or predisposition thereto. For instance, the term “diagnosis” as used herein substantially means any analysis for the presence or absence of a biological condition or biological trait. For example, the term “diagnosis” includes procedures such as screening for the predisposition for a condition or trait in the subject of interest, screening for a forerunner of condition or trait, screening for a condition or trait, and clinical or pathological diagnosis of a condition or trait, etc.

As used herein, the phrase “protein function data” means functional descriptors of proteins, i.e., information that describes the function, or lack thereof, of one or more proteins. The proteins, in some instances, are enzymes or structural proteins. Functional descriptors of such proteins include, but are not limited to, ubiquitination, alkylation, phosphorylation, disulfide bond formation, carbonylation, carboxylation, acylation, acetylation, glycosylation, prenylation, amidation, hydroxylation, adenylylation, and carbamylation. Additionally, protein function (or functional) data also means simply loss of function completely of the protein, or a certain degree of loss of function of the protein, group of proteins, or family of proteins, etc.

As used herein, the phrase “protein expression data” means the relative level of translation of an mRNA into protein. The relative level of translation activity for a particular mRNA is typically measured against industry-standard controls, such as the expression of one or more housekeeping genes, or alternatively measured against the expression level of wild type mRNA. Such data can include the translation activity from a single mRNA sequence, from a family of related sequences, or from an entire transcriptome, i.e., all mRNA sequences transcribed from a genome. Protein expression data, in some embodiments, also includes data and information characterizing various cellular translation regulators, including, for instance, ribosomes, microRNA (miRNA) or antisense RNA molecules, initiation factor molecules, and the like. Protein expression data can also include protein post-translational activity, such as truncation, processing of immature proteins to mature proteins by proteases, and the like. These translation variances will also in some cases alter the function and/or the protein activity.

As used herein, the phrase “gene expression data” means the relative level of transcription of a genomic segment of DNA into an mRNA molecule. The relative level of transcription activity for a particular gene is typically measured against industry-standard controls, such as the transcription of one or more housekeeping genes, or alternatively measured against the transcription level of wild type mRNA. Such data can include the transcription activity from a single gene sequence, from a family of related gene sequences, or from an entire genome, i.e., a transcriptome, all mRNA sequences transcribed from a genome. Gene expression data also, in some embodiments, include various control elements that govern the levels of mRNA transcripts in a cell at any given time, such as, for instance, enzymatic degradation of mRNA transcripts, enzymes controlling the rate of alternative splicing of mRNA, rate of intron/exon processing of mRNA transcripts, and action of other known transcription regulators that are in some cases proteins or enzymes that bind either the gene or mRNA to impact the rate of transcription of a gene or family of genes. Transcription regulators, in some embodiments, include transcription factors and other enzymes involved in the transcriptosome, i.e., polymerases, transcription factors, and the like.

In some embodiments described herein, cells of AIOs are referred to as being shaded or colored. In this context, the term “shade” or “shaded” means that the cell in question is darker or lighter in shade as compared to other cells, or as compared to a control cell, or as compared to pure white, i.e., no shading A color can be any number of shades. That is, while colors vary from blue to red to green to yellow, the intensity of the color, i.e., the shade of the color, can also vary from opaque to translucent. Thus, for any given color, there exists any number of various shades or intensities of that same color.

The term “artificial intelligence” (AI) as used herein means is the simulation of human intelligence processes by machines, especially computer systems. These processes include learning (the acquisition of information and rules for using the information), reasoning (using rules to reach approximate or definite conclusions) and self-correction. AI is sometimes referred to as “machine learning,” but machine learning is actually a subset of AI. AI is intelligence demonstrated by machines, i.e., any device that perceives its environment and takes actions that maximize its chance of successfully achieving a goal. The traditional problems (or goals) of AI research include reasoning, knowledge representation, planning, learning, natural language processing, perception, and the ability to move and manipulate objects. AIs used herein can be divided generally into classifiers and controllers. Classifiers, as used herein, use pattern matching to determine a closest match and are tuned (or taught) by analysis of examples to identify patterns or relationships between data points. The most common classifier used today is the artificial neural network (ANN).

As used herein, the term “artificial neural network” or ANN or neural net means a connection system, i.e., a computing system. An ANN is not a single algorithm, but instead is a framework for many different machine learning algorithms to work together to process data. By entering image data into an ANN, the ANN “learns” or is “taught” to identify images that contain characteristic signatures, or that do not contain the characteristic signature. After training the ANN, the ANN is then capable of identifying whether a given image or data set contains the characteristic signature or not. ANNs are well known in the art of AI. An ANN has also been described as “ . . . a computing system made up of a number of simple, highly interconnected processing elements, which process information by their dynamic state response to external inputs.” (See, Caudill, Maureen, “Neural Networks Primer: Part I,” AI Expert, 1989).

An artificial image object (AIO) is a visual representation of biological data. When the AIO represents genetic information, it is optionally termed herein a “genetic AIO.” Likewise, when the AIO represents protein information, it is optionally termed herein a “protein AIO.” AIOs in some embodiments include other data such as metabolomic data and microbiome data, and other types of data described herein.

A “cell” as referred to herein in reference to an AIO is a single unit addressable position within an AIO. An AIO is comprised of one or more cells Thus, an 8×8 AIO contains 64 individual cells addressable on a X vs Y axis and arranged in a box pattern in two dimensions. A cell within an AIO possess a specific address or coordinate designation of x vs y and is assigned a specific shade or intensity of color and/or a specific color, depending on the type of information encoded within that cell. Each cell therefore can encode multiple types of data, such as the expression level of a gene (shading intensity) for a specific gene sequence (color).

The term “training” or “learning” or “machine learning” as used herein in the context of artificial intelligence algorithms refers to a step in machine learning of an artificial intelligence algorithm. As known in the art, data is entered into an AI algorithm, for instance into its first layer, where the AI assigns a weighting to each input, noting how correct or incorrect it is, based on the task being performed, such as identifying or classifying an image. Thus, the term “machine learning” generally refers to computer-implemented and automated processes by which received data is analyzed by an AI algorithm to generate and/or update one or more models. Machine learning includes artificial intelligence, such as, in some embodiments, neural networks, genetic algorithms, clustering, or the like. Machine learning is performed in some embodiments by entering a training set of data into the AI algorithm. In such embodiments, the training data is used to generate the model that best characterizes a feature of interest using the training data. In some implementations, the class of features is identified before training. In such instances, the model is trained to provide outputs most closely resembling the target class of features. In some implementations, no prior knowledge is available for training the data. In such instances, the model discovers new relationships for the provided training data de novo. Such relationships include, for example, similarities between data elements such as shades, colors, and/or positions of cells, as is described in further detail below (See, for instance, Raschka, Sebastian, and Mirjalili, Vahid, “Python Machine Learning,” Packt Publishing, Ltd., Birmingham, U K, 2015, Chapter 2, pgs. 17-47, “Training Machine Learning Algorithms for Classification,” ISBN 978-1-78355-513-0) Training or learning can be performed either in a supervised mode or unsupervised mode.

The terms determine or determining encompass a wide variety of actions. For example, “determining” includes calculating, computing, processing, deriving, looking up, e.g., referencing a table, a database, or other data structure to find a specific data point or set of data points, ascertaining, and the like. Also, “determining” includes, in some embodiments, receiving, e.g., receiving information, accessing, e.g., accessing data in a memory, and the like. Also, “determining” includes, in other embodiments, resolving, selecting, choosing, establishing, and the like.

The phrase “genome-wide association study” (GWAS) as used herein refers to a method of evaluating the relationship between genetic markers or genetic variants and trait status. GWAS methodologies are commonly used for the discovery of genetic variants associated with a disease or trait. GWAS is also referred to herein and otherwise known as whole genome association study (WGA or WGAS). In such methodologies, a genome-wide set of genetic variants in different individuals are studied to determine whether any variant is associated with, or linked to, a specific trait. GWAS methodologies examine the DNA of individuals having varying phenotypes for a specific trait or disease. As the term implies, the GWAS methodologies examine an entire genome, and not just specific sections of a genome. GWAS methodologies employ control groups, case groups, and examine allele frequency amongst the groups to investigate any possible link or association between an allele and the trait. Examined data does not have to be genetic variants but can also include phenotypic data, including biomarkers and/or gene expression GWAS can also be performed using publicly available genetic variant information, such as that found at, for instance, the NCBI's dbGaP, or Database of Genotypes and Phenotypes GWAS results are also publicly available at, for instance, the U.S. National Human Genome Research Institute-European Bioinformatics Institute (NHGRI-EBI) catalog of published genome-wide associate studies, or GWAS Catalog.

The term “linkage disequilibrium” (LD) as used herein means a measure of non-random association of alleles at different loci in a given population. LD is commonly used to select genetic markers from different loci or to account for the correlation between different loci.

The term “optimizer” as used herein refers to a computer program that works in tandem with an AI program to update the model in response to the output of the loss function by combining the loss function and model parameters. Optimizer programs alter the model in such a manner to create the most accurate possible form by varying the weights assigned by the AI. That is, optimizer programs, within the context of AI, assist the AI to minimize (or maximize) an objective function, i.e., an error function, that is a mathematical function that is dependent on the model's internal learnable parameters used in computing target values from the set of predictors used in the model There are different types of optimizer algorithms, including first order optimizer algorithms and second order optimizer algorithms, as well as gradient descent algorithms, stochastic gradient descent algorithms, mini batch gradient descent algorithms, and the like. An exemplary optimizer is the TensorFlow optimizer. (See, Abadi et al., “TensorFlow: A System for Large-Scale Machine Learning,” USENiX Assoc., 12^{th} USENIX Symposium on Operating Systems Design and Implementation, OSDI, 16:265-283, 2016).

### Traits Based on Genetic and Protein-Based Variances

Over the last 50 years, genetic studies have demonstrated that many biological traits are influenced by genetic factors (Polderman et al., Nature Genetics, 47(7):702-709, 2015) including predisposition to many complex human diseases such as schizophrenia (Ronald et al., Human Mol. Gen., 27(R2):R136-R152, 2018; Blokland et al, Schizo. Bulletin, 43(4) 788-800, 2017, Sullivan et al., Arch. Gen. Psych., 60(12): 1187-1192, 2003), substance addiction (Vink, J. Studies Alcohol Drugs, 77(5):684-687, 2016; Dick, J. Studies Alcohol Drugs, 77(5):673-675, 2016; Yang et al., Mol. Psych., 21(8):992-1008, 2016), depression (McIntosh et al., Neuron, 102(1):91-103, 2019; Gómez-Coronado et al., J. Affect. Disord., 241.388-401, 2018), and other psychiatric disorders (Ludwig et al., Mol. Psych., 21(11):1490-1498, 2016; Gottschalk et al., Dialog. Clin. Neurosci., 19(2):159-168, 2017; Nievergelt et al, Biol. Psych., 83(10):831-839, 2018), as well as various cancers. These studies have shown that many different types of genetic variants contribute to such complex human traits. These genetic variants include single nucleotide variations (SNVs), copy number variations (CNVs), insertions and deletions (inDels), and other chromosomal rearrangements. Their effects on human traits vary, with very small effects for common SNVs to modest effects of rare SNVs, CNVs and inDels (Timpson et al., Nature Reviews, Genetics. 19(2):110-124, 2018; Wray et al., Cell, 173(7):1573-1580, 2018; Visscher et al., J. Am. Hum. Gen., 101(1):5-22, 2017, Jordan et al., Ann. Rev. Genomics Hum. Gen., 19.289-301, 2018). Although the effects of individual SNVs can sometimes be very small, collectively, SNVs can account for a large proportion of a biological trait of interest (Wray et al., Cell, 173(7).1573-1580, 2018; Khera et al., Nat. Gen., 50(9):1219-1224, 2018; Bipolar Disorder and Schizophrenia Working Group of the Psychiatric Genomics Consortium, Cell, 173(7):1705-1715, 2018). For a complex trait, the contributing variants could be in the hundreds, if not thousands, or more. On the other hand, for many complex human diseases, many risk variants have not been discovered.

Additionally, it has been discovered that traits are also linked to other factors, such as variations in gene transcription and translation, epigenetics, post-translational modifications of expressed proteins, proteomics, the microbiome, metabolomics, and other biological factors. (See, for example, Meaney, Michael J. Child Dev., 81(1):41-79, 2010; Albertin et al., Mol. Cell. Proteom., 12(3):720-35, 2013; Vaidyanathan et al., J. Biol. Chem., 289(5434466-71, 2014; Hanash S., Nature, 422(6928):226, 2003; Petricoin et al., J. Prof. Res., 3(4209-17, 2004; Ramezani et al., J. Am. Soc. Nephrol., 25(4):657-70, 2014; Cho et al., Nature Reviews Genetics, 13(4) 260, 2012; Orešič et al., Translational psychiatry, 1(12):e57, 2011, and Sellitto et al., PloS one, 7(3):e33387, 2012). Thus, it has been found that not only mutations in genes, but factors impacting genes after transcription also can have enormous impacts on biological traits, especially if that variance leads to depressed or lack of expression, depressed or lack of post-translational modification, etc. Such variances can lead to marked changes in protein function, and thereby cellular function, and ultimately organ function. As noted above, in addition to changes in genomic sequences described above, genes themselves can vary in their degree of expression, or epigenetics. These types of variations in epigenetics can lead to a titration of gene expression, aberrant gene expression, over-expression of genes in certain cells, under expression of genes, and even total lack of expression in cells where expression should be observed. Epigenetic variations in gene expression can be caused by nature, i.e., certain cells at certain times are pre-programmed to express certain genes only at certain times, or by nurture, i.e., environmental factors such as carcinogens, toxins, and other foreign substances can mildly or in some cases drastically alter gene expression. Epigenetic variations have been linked to numerous diseases and/or disorders (See, Simmons, Nature Education, 1(1):6, 2008; Moosavi et al., Iran Biomed. J., 20(5):246-258, 2016). These variations and changes in gene activity are caused by numerous molecular biology factors, such as DNA methylation, histone modification, RNA silencing, and such. Epigenetic variances have been linked to various cancers and psychological disorders (Id). These variances in epigenetic factors can be summarized in data sets. Many of these data sets are publicly available and individual subjects can be routinely tested for the presence of such epigenetic variances. For example, publicly available datasets can be found at The International Human Epigenome Consortium (IHEC) database, the NIH Roadmap Epigenomics Mapping Consortium, CEEHRC Platform, dbEM, DeepBlue, Epigenome Browser, ENSEMBLE, GenExp, and the Epigenome Atlas. After the gene is transcribed, the next step is translation of the RNA. There also occur variations in translation caused by protein function or dysfunction. (See, Taymans et al., Trends in Mol. Med., 21(8):466-472, 2015: and Scheper et al., Nature Rev. Gen., 8:711-723, 2007). Exemplary diseases or disorders linked to translation variances include Parkinson's Disease, X-linked dyskeratosis congenita, hyperferritinaemia, hereditary thrombocythaemia, X-linked Charcot-Marie-Tooth disease, and various forms of cancer caused by dysregulation of translation, such as melanoma, etc. (See, Id.). Such protein translation information can also be distilled to a database or dataset. One manner in which a dataset can be obtained from an individual characterizing translation within their cells is using a technique called ribosome profiling, which is based on deep sequencing of ribosome-protected mRNA fragments in a population of cells. (See, Wu et al., Database, 2018:bay074, 2018). Publicly available databases containing such ribosome profiling information include, for instance GWIPS-viz, RPFdb, TranslatomeDB, and the Human Ribosome Profiling Data viewer.

Additionally, after expression and translation of genes, the resultant protein can experience abnormal activity through variances in post-translational modification. Many post-translational modifications of proteins are known and well-characterized, including, for example, ubiquitination, alkylation, phosphorylation, disulfide bond formation, carbonylation, carboxylation, acylation, acetylation, glycosylation, prenylation, amidation, hydroxylation, adenylylation, citrullination, and carbamylation. A change in any of these activities within a cell can lead to changes in trait, susceptibility to disease or disorders, or a predisposition to contracting a disease or disorder, such as, for instance, rheumatoid arthritis, multiple sclerosis, Noonan syndrome, diabetes, Alzheimer's disease, heart disease, neurodegeneration, and cancer. (See, Gajjala et al, Nephrol. Dialysis Transplant., 30(11) 1814-1824, 2015, and Gyorgy et al., Int. J. Biochem. Cell Biol., 38(10): 1662-1677, 2006). Additionally, links between aging and aberrant post-translational modifications has been reported. (See, Santos et al, Oxid. Med. Cell Longev., 2017:5716409, 2017). Furthermore, disease-causing mutations have been linked directly to aberrations in post-translational modifications. (See, Li et al, Pac. Symp. Biocomput., 2010:337-347, 2010). Databases and datasets exist that are publicly available describing all known post-translational modifications for various genomes, such as the PTM Structural Database. Various techniques are well known and well developed to characterize such post-translational modifications in individuals. (See, Pascovici et al., Int. J. Mol. Sci., 20(16):1-30, 2019).

Thus, there are many genetic, epigenetic, and other (microbiome, metabolome, proteome) biologic variances that contribute to traits and disease. This information includes protein expression information (transcription, translation, post-translational modification) and protein function information. Protein expression and protein function information for any given individual subject can include hundreds, potentially thousands, or even hundreds of thousands, or millions of variations even between just two individuals. Fortunately, there exist databases cataloguing these variances, as noted above. These databases are publicly available and the volume of the data is growing daily upon completion of new studies and investigations in these areas. That is, all of these aberrations and variations in biological factors can be and are reduced to databases that are accessible by the public. Furthermore, any particular subject can be tested to determine the status of each of these variables, as noted in the studies cited above. Access to these data is obtained using methods known in the art. Alternatively, the trait data and information is obtained de novo, i.e., by using known assay methods to test and examine subjects possessing the trait of interest and subjects not possessing the trait of interest to generate proprietary databases of trait data. Tools and information are commercially available from multiple sources to obtain trait data from an unlimited number of subjects. Additionally, trait data is often held by private and/or public companies and either commercially available for a fee or by other means of acquisition.

Therefore, it follows that such biological data are able to be manipulated in any manner, and/or manipulated with any other type of (non-biological) data, as well as interpreted or investigated. Patterns in such data are routinely identified in the context of disease, as noted above. While some data is easily linked or connected to specific diseases, when examined in isolation, for example simply looking at SNV data alone, other data, or most other data, is simply reported and uploaded to the various publicly available databases without any such correlation study.

It therefore remains a large and challenging problem to not only catalog all of these data in some manner lending itself to interpretation, but also to somehow correlate these variances in biological data with specific diseases. Not only the volume data, but also the myriad different types of biological information (note above) create a daunting task for scientists and physicians looking to somehow correlate this information not only with disease susceptibility, but with diagnosis and actionable medical conclusions leading to treatment.

### Methods of Determining Presence of a Trait Using AIOs

Therefore, described herein are methods and systems for tackling the problem of generating actionable medical conclusions from not only voluminous biological variance data, but also data of different types. The methods and systems described herein synthesize the different types of biological data into a single set of actions that lead by design to actionable medical treatment of a specific subject in need thereof. These methods and systems are entirely scalable and able to process and analyze practically any volume of biological data submitted to the method steps described below.

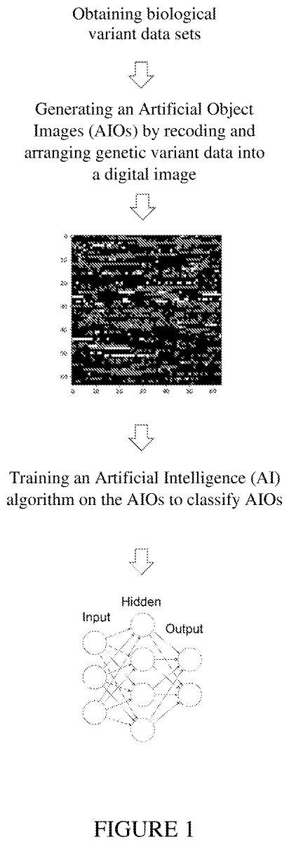

The general outline and flow of an embodiment of the methods described herein is depicted in FIG. 1, where it is shown that variant data is first collected (or obtained), which then leads to generation of Artificial Image Objects (AIOs). The AIOs are then used to train artificial intelligence (AI) algorithms specifically designed for pattern recognition such that the trained AI algorithm distinguishes between trait-containing AIOs and non-trait-containing AIOs.

The methods and systems described herein rearrange these data into specific geometric formations that lead to discovery of whether or not a specific subject is likely to possess a specific biological trait, i.e., the trait in question. These methods and systems are applicable to any subject of interest, so long as biological trait data is available for members of the same species of the subject. Thus, in one embodiment, a user may desire to determine whether a cow, chicken, goat, emu, or other agricultural or feedstock animal possess a specific biological trait. The methods described herein allow the user to make this determination based on biological trait data obtained from other members of the same, or similar, species as the subject in question. The methods described herein organize, arrange, catalog, and analyze biological trait data from positive and negative controls for the trait in question, i.e., biological trait data obtained from subjects of the same or similar species known to possess the trait, and subjects of the same or similar species known to not possess the trait, and thereby upon the testing of a specific individual subject allow for the determination of whether that individual subject possesses the trait in question.

Thus, in contrast to past correlative medical diagnostic methods that base medical decisions and actions on one or may be a couple of different biological variance data types, the present methods and systems are capable of synthesizing together in a unique combination biological data of any type, including genetic data, epigenetic data, proteomic data, microbiome data, metabolome data, and the like, into a single coherent, multidimensional and scalable process. In some instances of the present methods and systems, it is expected that this capability alone, i.e., the ability to combine and analyze vast amounts of different types of biological data, will lead to identification of correlations between biological trait variance data and symptoms of disease, susceptibility or predisposition to disease, identification of disease, and even real time diagnosis of disease conditions. Such output information, in some embodiments, is immediately medically actionable information leading directly to a known course of medical treatment to treat the identified disease, if any, in a specific subject.

The methods and systems provided herein achieve these results by performing the active steps described below. Briefly, these active steps entail obtaining trait data, as described and enumerated above, organizing these data into specific geometric patterns, and creating a“baseline” or basal level or control value for subjects who possess the biological trait in question and subjects who do not. The only requirement is that the biological trait data provided to the system and used in the methods, described herein, be readily segregated into data obtained from subjects who possess the biological trait in question (positive control data) and subjects who do not possess the biological trait in question (negative control data). As long as this minimum requirement is met for the database in question, the obtained data will be useful in the described methods and systems for achieving the stated goals.

### Obtaining Biological Trait Data

As a starting point in describing the methods provided herein, described here is one embodiment that is a simplified version of the methods and pertains to genetic data, and particularly pertaining to SNV data. In other embodiments of the methods described herein the biological trait data is not SNV data, but instead is data from any of the other categories of data described hereinabove.

In this embodiment, the biological trait data is SNV data, or information. The SNV information must be obtained from two sources. The first source is the subject in question, i.e., the subject for which knowledge of the presence or absence of the biological trait is desired. This subject is considered the test subject, i.e., the subject for whom the status of the biological trait is unknown. This is the first set of genetic variant data. As noted above, the methods described herein apply to any biological organism. For instance, in various embodiments of the methods described herein, the subject is a human, alpaca, cattle, bison, camel, deer, donkey, elk, goat, rat, mouse, horse, llama, mule, rabbit, pig, sheep, buffalo, monkey, ape, yak, dog, cat, chicken, fish, duck, goose, or hamster. In a particular embodiment, the subject is human.

The second source of biological trait data, or information, in this embodiment, is obtained from other individuals of the same species, or optionally a closely related species. This is the second set of genetic variant data. For instance, if the subject is human, then additional SNV information is obtained from other humans. In some embodiments, these other biological trait data from other individuals of the same or closely related species are publicly available. In another embodiment, the trait data is obtained de novo using known methodologies to assay subjects and individuals for the desired information. In other embodiments, the trait data is obtained from alternative commercial sources, i.e., from public or private companies who own the data and make the data available for a fee or through other means. As described above, there exist many publicly accessible depositories of SNV data from humans and other species. Thus, the second set of data in this embodiment is obtained from publicly available databases. As described above, these data act as the background against which the subject information is compared. That is, these data represent controls, both positive and negative, where the subjects from whom these data are obtained either possess the biological trait in question, i.e., positive control, or do not possess the biological trait in question, i.e., negative control. In this embodiment, the publicly available data already includes this additional piece of information, i.e., whether each subject individually possess the biological trait or not. This information is part of the SNV dataset, in this exemplary embodiment.

Both sets of biological trait data, SNV information in this embodiment, must be of the same type. That is, for every SNV genotype obtained from the individual test subject, the same SNV genotype must be provided by the individuals in the second set of SNV data. In another embodiment, not all SNV genotype are known for every position in the first or the second set of data. As explained above, SNVs occur throughout the genome of biological organisms. A single SNV therefore has both a position within the genome, and an identity, i.e., the identity of the genotype (AA, Aa, or aa, since there are two copies of the genome in each diploid individual) Thus, the identity of the genotype at each SNV position should be present in both the first and the second sets of genetic variant data. However, in some embodiments, the identity is not known for every SNV position in both sets of data. In such instances, the missing SNV (or other trait information) is addressed by standard methods of missing data replacement or interpolation. In one embodiment, the missing data is addressed by not including that specific SNV or CNV in the data sets, thereby reducing the total number of data points in each data set. In this embodiment, the method employs only the trait data that is held in common between the two data sets. In another embodiment, the missing data is filled in by any of the known methods, such as, for instance, simply using an average of the known possible values for the specific missing data points. In another embodiment, the missing data is imputed based on the known data and relationships between known data, using known methods. (See, for instance, Li et al., Annu. Rev. Genomics Hum. Genet., 10:387-406, 2009).

In an optional step, these data, both the first and second sets of genetic variant data, are culled, pruned, or otherwise filtered to create smaller subsets of the initial sets of data That is, in the genetic data embodiment discussed above, obtaining the two sets of genetic variant data is followed by a step of selecting specific SNVs from the two sets of data prior to performing the following active steps. The selection process creates two smaller subsets of genetic variant data corresponding to the two initial sets of genetic variant data.

The optional selection process is based on one or more additional points of data characterizing the two sets of data. In one embodiment, the selection process is based on an LD score (or gametic phase disequilibrium) That is, only certain SNVs in this embodiment are used in the following active steps and those certain SNVs are selected based on their linkage disequilibrium, as defined above. The individual LD score for each SNV is known since this information is generally available and accessible through the public databases containing the SNV data. Thus, in one embodiment, the biological variant data obtained in this step is first prned, selected, or screened and the resultant smaller subset of data is employed in the following steps described in further detail below.

In one embodiment, the biological trait information is SNV information. In another embodiment, only SNVs possessing a threshold LD value are filtered out of the initial set of SNV data and utilized in the following method steps. Linkage disequilibrium (LD) is a measure of the relationship among the variants on the DNA molecules. Thus, LD value is based on the non-random association of genotypes at two or more loci in a general population of subjects. By “association” it meant that the expected frequency of haplotype is not present. Factors that impact LD score include timing of the mutation event that generated the SNV, rate of genetic recombination, mutation rate, genetic drift, mating, population structure, genetic linkage, i.e., genetic distance between SNVs, and other factors of subject population history. A set of genotypes is entirely in equilibrium when they occur completely randomly in a given population of individuals. Disequilibrium occurs when the possible genotypes for any given SN V are not entirely random with respect to each other.

The threshold LD value is selected based on any number of factors known to one of skill in the art. For a more specific subset of SNVs, or loci, the LD value is selected as a numerical value ranging from 0.001 to 1.0 In one embodiment, the LD selected LD value threshold is 0.001. In another embodiment, the LD value threshold is 1.0. Any LD threshold value between these two numbers can be incorporated into the described methods directed to genetic variant data. In one embodiment, the LD value is 0.01, 0.02, 0.03, 0.04, 0.05, 0.06, 0.07, 0.08, 0.09, 0.10, 0.12, 0.14, 0.16, 0.18, 0.20, 0.22, 0.24, 0.26, 0.28, 0.30, 0.32, 0.34, 0.36, 0.38, 0.40, 0.42, 0.44, 0.46, 0.48, 0.50, 0.52, 0.54, 0.56, 0.58, 0.60, 0.62, 0.64, 0.66, 0.68, 0.70, 0.72, 0.74, 0.76, 0.78, 0.80, 0.82, 0.84, 0.86, 0.88, 0.90, 0.92, 0.94, 0.96, 0.98, or 1.0, or any number therebetween.

In another embodiment, the genetic variant data is further pruned based on one or more GWAS results. Genetic variants contributing to a trait have traditionally been discovered by association studies. Over the last decade, many genome-wide association studies (GWASs) have been conducted and a large number of risk variants has been discovered. (See, for example, Buniello et al., Nucleic Acids Res., 47(D1) D1005-D1012, 2019, and www ebi.ac.uk/gwas/). These discoveries provide great opportunities to develop strategies for personalized medical care. One application is to use these variants to model disease risks, facilitate accurate and objective diagnosis, and provide guidance for targeted and personalized treatment. Thus, in one embodiment related to the embodiment in which genetic variant information is used as the obtained two data sets described herein, and wherein the genetic variant information is SNV data, there is optionally an additional step of culling, pruning, selecting, or otherwise filtering the initial larger sets of SN Vs based on GWAS results.

As is known to one of skill in the art, GWAS results provide a characterization of the degree of association between a specific genetic variant, or set of genetic variants, and a disease. GWA studies typically focus on characterization of SNVs. GWA studies examine genetic variants across the entire genome of the subject being studied. The results of such studies are the identification of specific variants that occur more frequently in individuals known to possess the biological trait of interest. Thus, the selection of SNVs in the present methods described herein based on GWAS associations is a numerical cut-off selection based on the strength of the association of a particular SNV, or set of SNVs, in individuals known to have the biological trait of interest. The cut-off value in the context of GWAS results is arbitrarily selected. One such cut-off value is based on the association P value obtained as an output for SNVs in a GWA study. The exact threshold value for statistical significance of a specific genetic variant being correlated or associated with a specific biological trait or disease in a GWA study is often quoted as being 5×10⁻⁸ in the context of hundreds of thousands to millions of tested SNVs. However, other values higher or lower than this value are reported as possible thresholds.

Thus, in one embodiment, the methods described herein involve obtaining genetic variant data. In another embodiment, the genetic variant data are optionally pre-selected or filtered prior to carrying out any further steps in the method based on a characteristic of the genetic variants. In one embodiment, that characteristic is an LD value. In another embodiment, the characteristic is a GWAS association P value.

Additional embodiments of the methods described herein include obtaining additional data sets. These additional data include various “omics” data, such as, but not limited to, gene function and/or gene expression data, protein function and/or protein function data, proteomic data, metabolomic data, epigenetic data, microbiomic data, transcriptomic data, and the like. Thus, the described methods employ in certain embodiments not just genetic variant data, but instead employ other types of variant data as listed above. The only requirement is that the first set of data obtained from the subject of interest is identical in type to the second set of data obtained from the population of one or more second subjects so that for every set of data from the first subject, no matter what type of data it may be, there is obtained an equal quantity of similar types of data from the second set of subjects so that a direct comparison is made between the two sets of data.

Thus, in some embodiments, multiple sets of paired data are obtained and utilized throughout the described methods. The data pairs are always obtained from the first subject and from the population of second subjects, thereby providing paired data sets. For example, one paired data set is SNV data, another paired data set is CNV data, and yet another paired data set is protein post-translational modification data. In the following method steps described below, all three paired data sets are processed into the AIOs and analyzed by the AI, regardless of type of data, so long as there is data of the same type from both the first subject and the plurality or population of second subjects.

In other embodiments in which the biological variant data are not genetic variant data, such as, for instance, methods employing epigenetic data, metabolomic data, proteomic data, protein expression and/or functional data, etc., the two data sets are likewise optionally selected, pruned, filtered, or otherwise enriched based on similar concepts as described above, but for non-genetic variant biological trait information. Such selection criteria are known to one of skill in the art. For instance, in one embodiment wherein the biological trait information is phosphorylation or other post-translational modification, the selection criteria is optionally based on the degree of phosphorylation or other post-translational modification. For instance, it is known in the art that a single protein target can be phosphorylated multiple times. Each phosphorylation event for that individual protein target is known in certain instances to further modify the function of that target protein. Thus, in one embodiment, the selection criteria for further filtering of the initial two sets of biological variant data is the degree of phosphorylation. For instance, all data pertaining to proteins being phosphorylated less than once, twice, three times, four times, or six times or more, is ignored or removed from the data to create the filtered data sets that are utilized in the method steps that follow.

Similarly, it is well known that some protein targets are ubiquitinated. Some protein targets are further known to be ubiquitinated multiple times, creating either multiple ubiquitin sites on a single protein target, or a single ubiquitin site that becomes elongated into a chain of ubiquitin molecules, i.e., through a process of polyubiquitination Thus, in one embodiment, the methods described herein optionally include a further selection of the biological variant data for only those protein targets that are multiply ubiquitinated.

As described below, in some embodiments of the described methods, numerous sets of data are obtained for use in the following methods steps, thereby generating multi-dimensional AIOs by way of the described method steps. In such embodiments in which multiple types of data are obtained for use in the further method steps described below, multiple selection criteria are optionally imposed on the data to create multiple corresponding smaller subsets of variant information. The foregoing are merely exemplary embodiments of the methods described herein wherein numerous possible selection criteria are optionally imposed in the initial two data sets obtained for the further method steps described below. In one embodiment, no selection step is employed at all in the methods. In another embodiment only one selection criteria is employed in the method. In another embodiment, two, three, four, five, six or more selection criteria are imposed on the initial data sets to create secondary data sets upon which the remaining steps of the described methods are employed.

In particular embodiments of the methods described herein, wherein the variant data are post-translational modification data sets, these data sets are optionally pruned, trimmed, selected, and/or refined based on, for example, degree of post-translational modification. Thus, in embodiments in which the data sets contain information concerning the state of post-translational modification of proteins, the selection criteria upon which the optional selection step is based, are the degree to which the proteins are modified by one or more of the following: ubiquitination, alkylation, phosphorylation, disulfide bond formation, carbonylation, carboxylation, acylation, acetylation, glycosylation, prenylation, amidation, hydroxylation, adenylylation, and carbamylation.

Similar selection criteria are optionally imposed on the initial two sets of variant data even when the biological variant data are epigenetic data, microbiome data, metabolome data, gene expression data, or other protein expression and/or protein functional data. The selection criteria are based on the nature of the variant data. For instance, when the data are microbiome data, the selection criteria are, in some embodiments, is based on the presence or absence, or amount, of certain bacteria, or sets of bacteria, etc. For instance, when the data are epigenetic data, the selection criteria, in some embodiments, are based on the degree of methylation or other known epigenetic marker characteristic known in the art and previously characterized.

### Generating an Artificial Object Image (AIO)

In this exemplary embodiment, the SNV are converted to single pixel signals and a specific cell within a grid, and multiple SNVs are arranged into an Artificial Image Object (AIO) that is essentially a grid comprised of cells assigned in this manner. For example, as described above, most SNVs present as only two alleles, traditionally represented as “A” and “a.” Therefore, for each given SNV, there are three genotypes (AA. Aa and aa) or states for an individual with two copies of chromosomes. These three genotypes are, in this step of the described methods, assigned a pixel (or cell, the terms pixel and cell are used interchangeably herein) intensity.

The pixel intensity is arbitrarily selected to be 0, 154, and 254, respectively. However, any such pixel intensity can be selected as long as the imaging device is capable of distinguishing the difference in intensity value between the differently assigned intensities. Optionally, the intensity values are assigned to maximize the separation of the given genotypes Thus, intensity values assigned to the pixels depend on the machine that detects the intensity values in practice of the later method steps described hereinbelow.

In the prior method steps, two sets of data are obtained. The first variant data set is obtained from the subject in question, i.e., the test subject, for whom the status of the biological trait is not certain or not known. The second set of variant data is obtained from a population of the same, or closely related, species as the individual subject. Further, the two sets of data are of the same type, i.e., if SNVs are obtained from the subject in question in the first set of data, the second set of data will also be SNV information, and will contain the same SNVs as in the first set of data, i.e., from the same positions within the genome.

In the imaging step described here, a first AIO is generated based solely on the first set of variant data. Also in this step, a plurality of second AIOs are generated, each one based on an individual subject whose SNV are represented in the second set of data. These second AIOs are the “control” AIOs for which the presence or absence of the biological trait in question is known.

In one embodiment, the AIO is a 2-dimensional grid. In this embodiment, each box defined by the grid is assigned to a specific SNV, i.e., position on the genome. In this embodiment, the degree of intensity of shading of the cell assigned by a specific SNV is determined, as explained above, by the identity of the genotype for that SNV in that position. In this embodiment, the plurality of second AIOs are similarly generated, with each cell in the second AIOs corresponding to the same SNV as in the first AIO. Thus, each cell in this embodiment is assigned a specific SNV and the shading of each cell in all the AIOs is based on the genotypes found at that SNV position for a given individual.

In another embodiment, the cell is assigned a color. In this embodiment, the color is based on the genotype for the specific SNV assigned to that cell. For instance, where the genotype possibilities are AA, Aa, and aa, the assigned colors are green, blue, and yellow, respectively. However, in other embodiments, other colors are selected for the various genotypes for each cell. The only requirement is that the machine that detects the colors is capable of detecting the differences in the colors of each cell.

In other embodiments, where the variant data are not genetic variant data, the cells are likewise assigned based on any specific variant information present in the obtained data sets. Likewise, the colors or shades of the cells assigned based on these data are chosen based on the type of data represented by the AIO. For instance, of the variant data is post-translational modification, for instance phosphorylation, the assigned cell is based on the specific protein target that is phosphorylated (or not phosphorylated). Further, in this embodiment, the shade/intensity and/or color of the cell is optionally based on the degree of phosphorylation, etc. In another embodiment, the post-translational modification is one or more of ubiquitination, alkylation, phosphorylation, disulfide bond formation, carbonylation, carboxylation, acylation, acetylation, glycosylation, prenylation, amidation, hydroxylation, adenylylation, and carbamylation. Likewise, the assigned cells are based on the identity of the target protein that is modified by these post-translational modifications, and the color, intensity, and/or shading of the assigned cell is based at least in part on the degree of post-translational modification.

In this step of one embodiment of the described methods, the data point obtained from the first or second sets of data assigned to a specific cell is arbitrary. That is, the AIO in some embodiments is a square or rectangular grid, for example, and the coordinate system of the grid define a specified number of cells. Each cell is then assigned to a specific data point within the two sets of variant data. This assigning step within the described methods is arbitrary in one embodiment. Thus, in embodiments in which the variant data are genetic variants, and the genetic variants are SNVs, for example, any given cell is assigned to any given data point or specific SNV, in no particular order or orientation. The only requirement in this embodiment is that the assigned data point for each cell remain identical between the two data sets and therefore between the first AIO and the plurality of second AIOs such that at any given cell position, the same SNV data is reflected across all AIOs for whichever individual data set the AIO is based upon.

In another embodiment of the described methods, the assignment of variant data to cells is strictly ordered. For example, in the embodiment in which the variant data is SNV information, the first SNV appearing on the first chromosome closest to a particular end of the chromosome, i.e., closest to the telomere sequences, i.e., the position furthest upstream within the chromosome, is assigned to cell position 1,1 in the AIO. In another embodiment, the cell positions are assigned specifically based on chromosome numbering and optionally distance from telomere sequences, or ends of chromosomes, such that in the x direction from left to right, distance from telomere sequence increases, and in the y direction the chromosome number increases from top to bottom, for example. This is just one embodiment of the variety of ways in which the cells within the AIO are, in some embodiments, specifically ordered based on the type of variant data that form the basis of the generated AIO.

It follows then that each AIO generated in this method step is specific to each individual subject because each individual subject possesses a unique biological profile, e.g., a unique set of genetic variants, epigenetic markers, a unique metabolome, a unique proteome, a unique transcriptome, and the like. In embodiments in which the variant data are genetic variants, and wherein the genetic variants are SNV, since each SNV occupies only a specific cell, an AIO can easily handle millions genetic markers, significantly improving the capacity and efficiency of genetic analysis. Thus, in one embodiment, the AIO comprises a million or more cells. In another embodiment, the number of cells is less than a million. In other embodiments, the number of cells is 500,000, 600,000, 700,000, 800,000, 900,000, 1 million, 1.5 million, 2 million, 2.5 million, or more than 3 million.

In some embodiments of the methods described herein, the AIOs are 2-dimensional AIOs. That is, the AIO represents a grid system comprised of cells, each cell assigned to a specific data point within the set of biological variant data. In one embodiment, the AIO is one-dimensional, e.g., a line or broken line, optionally including different colors and/or sizes, etc. such that it can be used by an AI algorithm such as natural language processing (NLP). In other embodiments, the MO is more than 2-dimensional, i.e., comprises additional dimensions. In these embodiments, the AIO is generated based on not just one set of variant data, but more than one set of variant data. In such embodiments, the AIO is 3, 4, 5, 6, 7, 8, 9, or as many as 10 dimensions. In such embodiments, each “dimension” of the AIO above two dimensions also comprises individual cells, and each individual cell is also assigned to a specific data point within the additional set(s) of variant data.

In multi-dimensional AIOs, in one embodiment, the first two dimensions of the AIO define an arrangement of cells, as described above, wherein each cell is assigned a specific intensity or shade, as described above, based on the specific data point assigned to that specific cell within the AIO for the specific individual subject. In one embodiment, a third dimension is added to the 2-dimensional AIO by also assigning each cell a specific color, in addition to the intensity or shade. Thus, the third dimension in this embodiment represents a color. In such embodiments, for example, the third dimension is generated based on an additional type of data within the first and second data sets. Thus, in addition to the first type of data, the 3-dimensional AIOs are generated based on at least two different types of variant data, reflected in one single AIO. As an example, the second type of data is copy number variant (CNV) data. Thus, each cell in this embodiment of 3-dimensional AIO is colored and shaded based on both a specific CNV of a specific gene and a specific SNV genotype for that individual subject upon which the AIO is based.

In another embodiment, the AI) comprises more than two dimensions, as described above, including a fourth dimension. In this embodiment, the fourth dimension is based on a third type of variant data. The third type of variant data is, for example, protein function and/or protein expression data. Visually, one can think of this additional dimension as a three-dimensional graph, wherein third type of variant data is represented by additional cells present in the z direction in the above-mentioned AIO grid layout, for example.

In a further embodiment, the AIO comprises three or more dimensions, with each dimension correspondingly generated by a further different type of variant data. The additional dimensions are optionally based on assignment of different colors, different shading and/or intensity, and/or different patterns represented in each cell, such as cross-hatching, dots, stripes, or any other machine-recognizable pattern. In some embodiments, the patterns assigned to each cell are also assigned specific colors, with each color corresponding to a specific data point or status found in the additional type of variant data set. In another embodiment, each data type is incorporated into a separate AIO and the determination of whether the trait is present or not depends on analysis of multiple AIOs.

### Training Artificial Intelligence (AI) Algorithms on the AIOs

The methods described herein include steps of processing the AIOs generated in the previous steps by submitting the AIOs to analysis by artificial intelligence (AI) algorithms. Processing of the AIOs by an AI designed to recognize patterns generates rules within the AI governing spatial relationships between individual cells of AIOs along with the colors and/or intensity/shading of each cell, in any number of dimensions used to generate the AIO (as explained above). With these learned spatial relationships incorporating colors and/or shading/intensities, the AI processing learns which AIO patterns indicate the presence of the biological trait in question and which patterns are not indicative of the presence of the biological trait in question.

As noted above, in one embodiment of the described methods, the biological variant data is genetic data, and the genetic data is SNV data. In such an embodiment, because each pixel in each AIO is assigned to a specific SNV, the spatial and color/shading/intensity relationships among the various cells represent an index of the genetic relationship between the SNVs. This index not only includes the spatial relationship between multiple SNVs as well as any additional data set information incorporated into the AIO, such as selection information, e.g., LD or GWAS selection, as well as other types of data such as CNV data, or gene expression and/or gene function data, or protein expression and/or protein function data.

From a genetic association perspective, such AIOs represent single and multi-point associations, as well as single and multi-point interactions. Therefore, the patterns found in a AIO by the AI algorithm are associated with the trait of interest influenced by the genetic factors present in the variant data sets. The pattern recognition performed by the AI is then utilized to build a classification structure of each AIO type.

AI algorithms are well known in the art. In some embodiments, the AI algorithm is a machine learning (ML) algorithm. In other embodiments of the described methods, the AI algorithm is an artificial neural network (ANN).

In some embodiments, the ML is one or more of the following exemplary MLs known in the art, such as attention mechanisms & memory networks, Bayes theorem & naive Bayes, decision trees, eigenvectors, eigenvalues, evolutionary & genetic algorithms, expert systems/rules engines/symbolic reasoning, linear regression and ordinary least squares regression, generative adversarial networks (GANs), graph analytics, support vector machines, logistic regression, LSTMs and RNNs, Markov Chain Monte Carlo methods (MCMC), ensemble methods, random forests, reinforcement learning, word2vec and neural embeddings in natural language processing (NLP), clustering algorithms, principal component analysis, singular value decomposition, and independent component analysis.

Additionally, in another embodiment, the AI algorithm is an artificial neural network (ANN). ANNs of varying types are known in the art and available to the public that are capable of performing pattern recognition tasks required by the methods described herein. Such ANN include, but are not limited to, for instance, the following types of ANN: convolutional neural network (CNN), a deep learning neural network (DNN), a deep, highly nonlinear neural network (NNN), a developmental network (DN), a long short-term memory network (LSTM), a recurrent neural network (RNN), a deep belief network (DBN), large memory storage and retrieval neural network (LAMSTAR), deep stacking network (DSN), spike-and-slab restricted Boltzmann machine network (ssRBM), or a multilayer kernel machine network (MKM).

In a particular embodiment of the described methods, the AI algorithm employed in the training steps and analysis steps is an algorithm capable of complex pattern recognition and able to distinguish between various AIOs from subjects who possess the biological trait of interest and subjects who do not possess this trait.

As explained above, in the training step, the generated AIOs from the second set of variant data is first subjected to AI analysis to teach the AI program to recognize patterns that indicate the presence of the trait of interest and patterns that indicate that the trait of interest is not present. The second set of variant data comprises data from a plurality of subjects that are known to either possess the trait of interest (positive controls) or not possess the trait of interest (negative controls). This additional information, presence or lack thereof of the trait of interest, is also submitted to the AI program. This information, along with the data depicted in the generated second set of AIOs, teaches the AI to distinguish between AIOs possessing the trait and AIOs that do not possess the trait.

As known in AI theory, the amount of time and/or amount of data needed to fully enable an AI to distinguish between the presence of a pattern or lack of a pattern, or to identify a particular pattern, e.g., a picture of a cat, varies depending on the degree of certainty imposed on the AI program. If a low degree of certainty is imposed, the training step will require less time, and conversely if a high degree of certainty in the ultimate determination step is desired, a relatively longer training time, and higher number of training samples, will be needed to achieve that goal.

Other commonly known concepts of AI programming are not included here but nonetheless are contemplated, such as the number of training steps, algorithm convolutional layers, etc. These variables are known and in various embodiments of the described methods are able to be routinely optimized to obtain the best results. Additionally, it is well known that even given an excessive amount of time and data, it is unlikely that any pattern recognition AI algorithm will be capable of determining the presence of a pattern with 100% accuracy. Graphic plots of the AI accuracy vs. the number of training steps typically plateau at a value less than 100%. Thus, it is common practice to stop training the algorithm when this plateau is reached. Further, the known variables for algorithm training that are routinely optimizable and known in the art and contemplated herein are in some methods varied depending on the amount of computing power available, the amount of time available to the user, and/or the amount of data or AIOs generated therefrom available for analysis and training by the AI. That is, one of skill knows how to optimize the AI based on these factors and such optimizations are contemplated herein and within the scope of the described methods.

It is contemplated herein, and within the scope of the presently described methods, that the amount or number of individual data points with each of the first and second variant data sets, is itself variable. Likewise, the number of subjects for which variant data is available for the second set of data (controls), will determine the number of steps of training required by the AI to achieve pattern recognition within the desired accuracy threshold. That is, if the variant data is CNV or SNV, it is known that for certain traits, there may be only a certain amount of publicly available SNV or CNV data capable of being analyzed by the present methods. While an unlimited number of data points is possible to be analyzed by these methods given an unlimited amount of time and/or computing power, less variant data may be available for certain traits or diseases. What is required to achieve the methods described herein is an amount of variant data sufficient to allow the requisite amount of training steps on the generated AIO necessary to achieve pattern AIO recognition by the AI within the desired degree of accuracy. Of course, if a lower degree of accuracy is sufficient for pattern recognition by the AI, then less variant data will be required.

That is, if a high degree of accuracy is desired, then more variant data will likely be required both for the first data set (test) and the second data set (control). However, one of skill is able to routinely optimize the variables of AI training to achieve the desired outcome in most situations depending on the number of data points in any given data set, the number of subjects for which variant data is available in the second data set, and the number of different types of data sets, incorporated into the AIOs.

Further contemplated herein are the use of various optimizers known in the art of AI technology. Optimizer programs provide additional functionality to the AI to allow further refining and tuning of the AI learning process, thereby achieving results with higher accuracy or more quickly based on a relatively smaller amount of data, etc. An exemplary optimizer is the TensorFlow optimizer. (See, Abadi et al., “TensorFlow: A System for Large-Scale Machine Learning,” USENIX Assoc., 12^{th} USENIX Symposium on Operating Systems Design and Implementation, OSDI. 16:265-283, 2016).

Analyzing AIOs with the Trained AI and Determining Whether the Trait of Interest is Present in the Subject

In an additional step of the methods described herein, after the AI is sufficiently trained to recognize or distinguish the AIO pattern for trait-containing individuals and non-trait-containing individuals, includes processing of the first AIO from the test subject by the AI. In this step, the AIO of the subject of interest is submitted to the AI for processing and pattern identification.

In this step, if the AI recognizes the trait pattern in the first AIO, it is then concluded that the subject of interest possesses the trait of interest. As noted above, such determinations are made by the AI based partly on the degree of accuracy with which the determination is desired by the user. Conversely, in this step, if the AI does not identify the trait pattern in the first AIO, then it is concluded that the subject of interest does not possess the trait of interest.

Based on this determination and conclusion, further active steps are contemplated. For instance, in one embodiment, the variant data includes SNV and/or CNV variant data. Analysis of the corresponding genetic AIOs based on these SNV and CNV data by the AI in this embodiment then achieves determination of the presence or absence of the trait of interest in the subject of interest. In some embodiments, as described above, the trait of interest is a disease, or susceptibility to or predisposition for a disease, or other biological trait.

In an embodiment in which the trait of interest is, for example, a cancer, then following determination by the AI of the presence of the cancer trait, the subject of interest is further prescribed medical treatment by an attending physician. In some embodiments, the medical treatment is preventative. In some embodiments, the trait of interest is, for example, a carcinoma, sarcoma, myeloma, leukemia, or lymphoma. The prescribed medical treatment, in some embodiments, is a cancer vaccine or other preventative treatment to protect the subject from being susceptible to the cancer.

In another embodiment, the trait of interest is one or more mental disorder or condition or illness. In certain embodiments, the one or more mental illnesses comprises one or more of a neurodevelopmental disorder, schizophrenia, bipolar disorder, anxiety disorder, trauma related disorder, dissociative disorder, somatic symptom disorder, eating disorder, sleeping disorder, impulsive/disruptive/conduct disorder, addictive disorder, neurocognitive disorder, or a personality disorder. In such embodiments, the method optionally includes an additional active step of prescribing treatment for the identified trait. Such treatment includes, for instance, prescription of one or more active pharmaceutic agents (API), scheduling of regular counseling sessions, and the like. In such embodiments, the identified biological trait is not manifest at the time the method is conducted, but instead the biological trait is a susceptibility or predisposition to the mental illness, disorder, or condition. In such embodiments, the method optionally includes the further active step of prescribing preventative counseling and/or prescription of preventative API to the subject of interest.

### Systems for Determining a Trait Using AI

All of the embodiments of the methods described herein are contemplated to be embodied in, and partially or fully automated by, software code modules executed by one or more computers specifically designed for the purpose of conducting the described methods. For instance, the specifically designed computers include such elements as processors, video screens for visualization of data and results, as well as memory devices containing the specialized software code modules necessary for conducting the above-described methods. For instance, the memory devices in some embodiments contain software code modules that embodies the AI and various appurtenant programs, such as optimizers, etc., useful for running the AI algorithm software and selecting the variables discussed above pertinent to the AI algorithm, such as number of steps and layers and the like. Further, the computer memory devices will comprise biological variant data, or are equipped to receive such data, and store these data along with the code modules. Such computers optionally also include ethernet cards and other devices known in the art for connecting to the internet and downloading biological variant data from various database sources identified in the above descriptions. Additionally, such computers optionally comprise keyboards and other devices useful for users to interact with and manage the computer before, during, and after performing the methods described herein.

Software code useful in conducting the methods described above and embodying the AI code modules include, for instance, Python, LISP, GO, Prolog, C, C++, Scala, R, Java, and the like known in the art to be capable and useful in coding AI programs and modules. In one embodiment, the code used to program the AI is Python.

Memory devices are known in the art, such as hard drives, solid state memory, optical discs, and the like. Also known are various non-transitory computer-readable media devices capable of storing and executing the AI programs and other software modules described above.

That is, each of the processes, methods, and algorithms described in the preceding sections are in some embodiments embodied in, and fully or partially automated by, code modules executed by one or more computer systems or computer processors comprising computer hardware and computer-readable medium. Examples of computer-readable mediums include, for example, read-only memory, random-access memory, other volatile or non-volatile memory devices, compact disk read-only memories (CD-ROMs), magnetic tape, flash drives, and optical data storage devices. Coded modules also include, in some embodiments, software modules that generate visual images, such as the above-described AIOs, upon submission of the requisite data sets Thus, in addition to data processing modules, there are in some embodiments AI module(s) and one or more imaging modules that calculate, generate, and/or display the AIO for a use to visualize. Optionally such imaging modules include specific software and code that allows the user to print copies of the images or save electronically the AIOs for future use and presentation in various forms of media. The systems and modules are also in some embodiments transmitted as generated data signals (for example, as part of a carrier wave or other analog or digital propagated signal) on a variety of computer-readable transmission mediums, including wireless-based and wired/cable-based mediums, and take a variety of forms (for example, as part of a single or multiplexed analog signal, or as multiple discrete digital packets or frames). The processes and algorithms are in some embodiments implemented partially or wholly in application-specific circuitry. The results of the disclosed processes and process steps are in some embodiments stored, persistently or otherwise, in any type of non-transitory computer storage such as, for example, volatile or non-volatile storage.

Thus, in some embodiments, the systems contemplated herein, are specialized for performing the methods described herein. In some embodiments, the systems include one or more user interfaces A user interface (also referred to as an interactive user interface, a graphical user interface or a GUI) refers in some embodiments to an interface, optionally web-based, including data fields for receiving input signals or providing electronic information and/or for providing information to the user in response to any received input signals. A GUI is implemented, in whole or in part, using technologies such as HTML, Flash, Java, net, web services, RSS, or other known programming language that serves the same purpose. In some implementations, a GUI is included in a stand-alone client (for example, thick client, fat client) configured to communicate (e.g., send or receive data) in accordance with one or more of the aspects described.

In a further embodiment of the described methods and systems, there are specialized systems to carry out the described methods that optionally comprise a specialized computer chip, graphics card, memory chip, or other non-transitory memory device, that is specially designed to perform the described methods, i.e., that provide additionally computing capacity above that normally found in a typical computer chip. The additional computing capacity is used, for example, in generating the multiple AIOs described above. Such specialized chips possess, additionally, programming modules and other scripts or software enabling rapid generation of large numbers of AIOs and analysis of the same. Such specialized chips are, in some embodiments, equipped with circuitry and other components designed to enhance, make more efficient, and/or more quickly generate, analyze, and process visual information, such as AIOs. Such systems optionally further comprise specially designed image processing boards, image capture boards, and the like for performing the above-described methods. Such specialized components are, in one embodiment, commonly referred to as system on a chip or SoC and comprise such components as a central processing unit (CPU), memory, input/output ports, secondary storage, as well as processors capable of processing digital, analog, mixed-signal, and other signals as may be required by the described methods. In such embodiments, the specialized components include those useful in, and capable of efficiently performing, 3D modeling and rendering and in some embodiments include software specifically designed to aid in 2D and/or 3D modeling and/or rendering of AIOs.

Finally, contemplated herein are systems comprising the above-identified components of computers, software, memory devices, data, AI components and algorithms, and visualization screens.

Further modifications and alternative embodiments of various aspects of the methods and systems described herein will be apparent to those skilled in the art in view of this description. Accordingly, this description is to be construed as illustrative only and is for the purpose of teaching those skilled in the art the general manner of carrying out the disclosed methods and systems. It is to be understood that the forms of the disclosed methods and systems shown and described herein are to be taken as examples of embodiments. Elements and materials may be substituted for those illustrated and described herein, parts and processes may be reversed, and certain features of the disclosed methods and systems are capable of being utilized independently, all as would be apparent to one skilled in the art after having the benefit of this description of the disclosed methods and systems. Changes may be made in the elements described herein without departing from the spirit and scope of the disclosed methods and systems as described in the following claims.

All of the references cited above, as well as all references cited herein, are incorporated herein by reference in their entireties. The following examples are offered by way of illustration and not by way of limitation.

### EXAMPLES

### Example 1: Materials & Methods

All experiments were performed in silico on a Puget Systems computer with 128 GB of RAM, Intel Xeon W-2145 CPU processor and NVIDIA® GeForce RTX2080 Ti GPU running Microsoft Windows 10.

Human genetic data were obtained from public databases as noted below.

Series GSE71443 SNV Array Dataset: The GSE71443 dataset was accessed from the U S National Institutes of Health (NIH), National Library of Medicine (NLM), National Center for Biotechnology Information (NCBI), Gene Expression Omnibus (GEO) Database (ncbi.nlm nih.gov/geo/query/acc.cgi)acc=GSE71443). This dataset contains genotypes and gene methylation data from 203 unique human individuals, of them, 75 were healthy control individuals, 63 were diagnosed with schizophrenia, and 65 were diagnosed as bipolar disorder patients. Subjects in this dataset were all individuals of European ancestry. Both genotype and methylation data were obtained using the Affymetrix Genome Wide Human SNV 6.0 Array (“Affymetrix 6.0”). (Affymetrix, Inc., which is now Thermo Fisher Scientific, Santa Clara, Calif., US). In this genetic dataset, brain samples were interrogated twice on Affymetrix SNV 6.0 microarrays: first, regular SNV genotyping was performed following the manufacturer's protocol, and second, allelic differences in DNA methylation was investigated by enriching the unmodified DNA fraction using DNA methylation-sensitive restriction enzymes. However, only genotype data were used in the following experiments. Array intensity data are available as .CEL files, a format created by Affymetrix DNA microarray image analysis software containing the data extracted from probes on an Affymetrix GENECHIP™. As known in the art, .CEL files are processed by software algorithms and visualized on a 2D grid as part of an overall genome experiment. Array intensity data (.CEL files) were downloaded from the GEO website and processed by genotyping with Genotyping Console software (Version 4.2). (Thermo Fisher Scientific, Santa Clara, Calif., US). Genotypes produced from Genotyping Console were exported into pedigree (.PED) file format for downstream analyses, described hereinbelow. .PED files are tabular text files describing meta-data about familial samples. (See, Chang et al., Gigascience. 4:7, 2015).

Series GSE81538 and GSE96058 RNA Genetic Datasets: These two human RNA-seq datasets were also downloaded from GEO database (ncbi.nlm.nih.gov/geo/query/acc.cgi?acc=GSE81538, and ncbi.nlm nih.gov/geo,queiy/acc.cgi?acc=GSE96058, respectively). These two datasets came from a study that used whole genome transcription to classify breast cancer (BC) tumors into subtypes. (Brueffer et al., JCO Precision Oncology. 2:1-18, 2018). The GSE81538 database includes expression data of 405 BC tumors with extensive immunohistochemistry characterizations by three independent pathologists, including subtype classifications for estrogen receptor (ER), progesterone receptor (PgR), human epidermal growth factor receptor (HER2). Ki67 antigen, Nottingham histologic grade (NHG) and PAM50 classifications (subtypes). GSE96058 is a prospective study of Swedish women (n=3,273) with similar gene expression and phenotype measures. For both datasets, gene expression levels were assessed with paired-end RNA sequencing. The objective of the study was to evaluate whether the expression of specific genes could be used as biomarkers to classify BC tumors and the trajectory of disease course (prognosis).

Series GE025016 SNV Array Dataset: This dataset was downloaded from GEO database (ncbi.nlm.nih.gov/geo/query/acc.cgi?acc=GSE25016). This is a data set from a lung cancer study with 155 squamous-cell lung cancer samples, 77 adenocarcinoma of the lung samples, and 59 normal samples. The study was designed to interrogate CNV at the fibroblast growth factor receptor (FGFR) gene and its relationship with therapeutic effects using the Affymetrix® SNP 6.0 array (Weiss at al., Sci Trans. Med, 2(62):62-93, 2010). The available data at the Geo Database include a raw intensity file (.CEL) and genotype files for each of the normal, adenocarcinoma, and squamous cell groups. The genotype data were used to build models to classify the normal samples and lung cancer subtypes.

### Example 2: Recording Genetic Variants to Genetic Images for Analysis

For most SNVs, there are two alleles, A and a. Since humans have two chromosome copies, therefore, for a given SNV, there are 3 genotypes, AA, aA, and aa. In most gene association analyses, SNVs are analyzed individually. In risk assessment and prediction models, SNVs are also entered into the models as individual terms, and the interactions among SNVs are not modeled. With polygenic analysis, only a single score is modeled. There are many disadvantages with these approaches. When SNVs are modeled individually, there is a limit on how many SNVs can be included in the model for a study with a given sample size. It is unrealistic that hundreds of thousands or more SNVs can be modeled effectively with this typical analytic approach at this time.

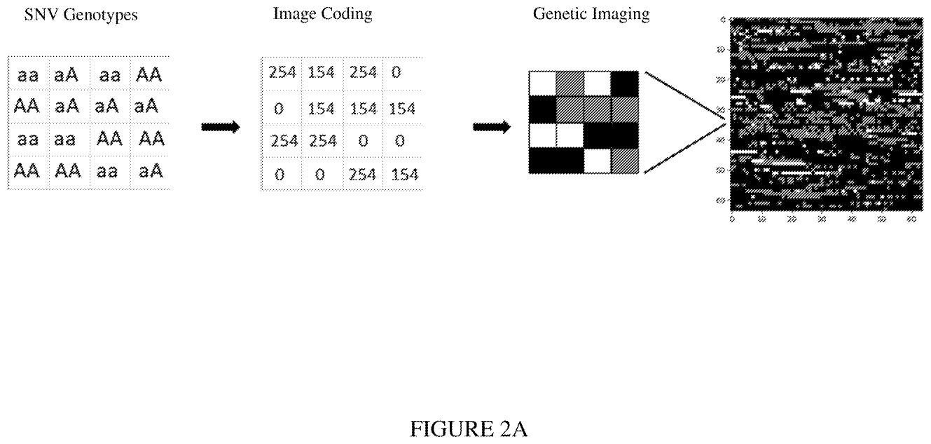

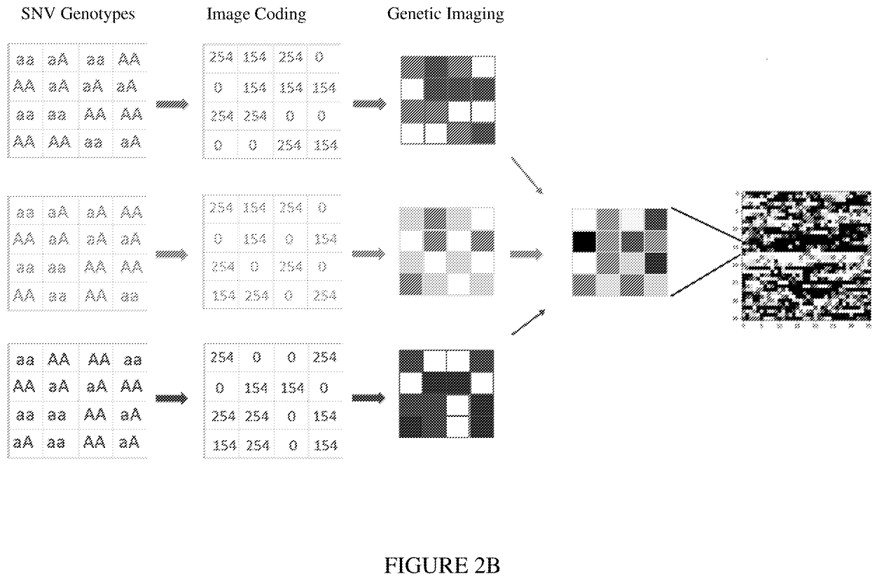

To improve the efficiency of SNV analysis, a new algorithm was developed to analyze the relationship between a group of SNVs and a trait of interest. The algorithm is inspired by recent advancement of AI algorithms in image recognition (classification) and prediction. In image analysis, two dimensional patterns can be learned through deep neural network (DNN) and convolutional neural network (CNN) (Chen et al., In 2015 3rd IAPR Asian Conference on Pattern Recognition (ACPR), pp. 695-699, 2015; Abadi et al., USENIX Assoc., 12th USENIX Symposium on Operating Systems Design and Implementation, 265-283, 2016) In these analyses, intensity signals in an image are processed and analyzed pixel by pixel. In the AIO analysis, SNV data are recoded and rearranged in a specific procedure and converted into an image. In the new coding algorithm, each SNV is treated as a pixel, and its value can take one of the three possible genotypes A collection of selected SNVs can be arranged as an image (FIGS. 2A and 2B). In this arrangement, the physical distance and relationship of SNVs on chromosomes can be indexed by the pattern formed by these SNVs because each SNV occupies a specific address in the image, and the spatial relationship between any two pixels is clearly defined. The image formed will allow not only the analysis of the relationship between a single SNV and the trait of interest (analogous to traditional single point association analysis), but also the identification of the complex relationship between a specific pattern made of multiple SNVs and the trait (multipoint interaction and association).

The number of SNVs included in a AIO and which SNVs are to be included in the AIO varies depending on the objectives of the analyses and computational resources. In AIO analysis, SNVs can be coded as a two dimensional or three-dimensional image. For example, in a two dimensional gray scale image, SNVs are coded as the following: for a given SNV with a G/A variant, the image code for an individual with the G/G genotype (major allele homozygote) would be assigned the value of 0; for an individual with the G/A genotype (heterozygote), the code value assigned is 154; and for an individual with the A/A genotype (minor allele homozygote) the value of 254 is assigned Although the values of 0, 154, 254 are chosen arbitrarily, they are chosen for easy visual distinction in a gray scale image. Other values can be used as long as the three genotypes are distinct. The images produced from this procedure of SNV coding and arrangement are referred to as artificial image objects (AIOs).

An exemplary AIO of two-dimensional gray scale is shown in FIG. 2A. For an AIO of multiple colors, the primary colors (red, green, and blue) are treated as the third dimension, and each color forms a separate layer (colors are represented as various shades of gray). For each of these three colors, the SNV genotypes can take the values as in the gray scale image. The three colored layers form a colored AIO (FIG. 2B). With a three-dimensional image coding, three times more SNVs can be coded in an image than a gray scale image with the same dimensions.

### Example 3: Binary Classification with GWAS Identified SNVs-Distinguishing Patients of Schizophrenia from Healthy Controls Using a 3-Color Coding Scheme

The GSE71443 dataset has 203 subjects, of them, 75 are healthy controls, 63 are schizophrenia patients, and 65 are bipolar disorder patients. In this example, only the healthy subjects and schizophrenia patients were used, i.e., N:=75+63 Raw data downloaded from the GEO website included intensity data and the subject's demographic and diagnostic information. Genotyping Console software (Version 4.2) was used to process the intensity file and make genotype calls. (Thermo Fisher Scientific, Santa Clara, Calif., US). The platform used for GSE71443 genotyping was Affymetrix 6.0 Array, which had 900,660 SNVs. (Thermo Fisher Scientific, Santa Clara, Calif., US).

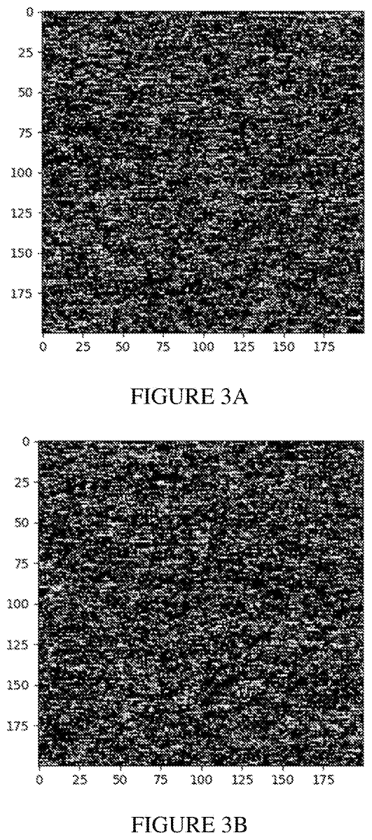

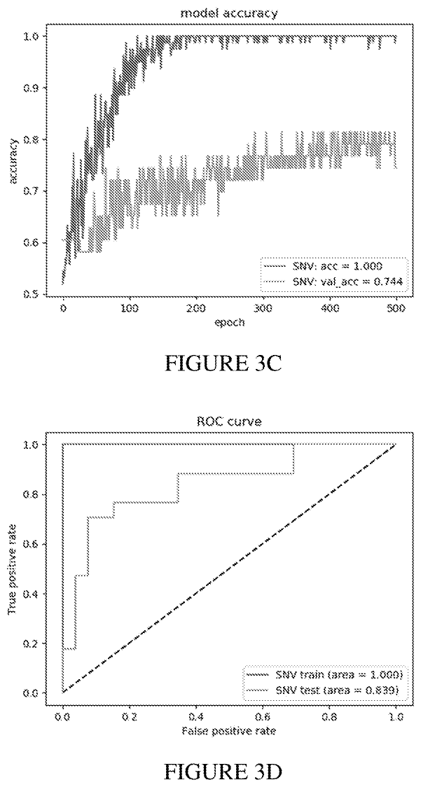

In this example, the objective was to classify the two groups of subjects included in the GSE71443 dataset, i.e., healthy controls and schizophrenia patients, each with SNVs identified by GWAS. Towards this goal, SNVs relevant to schizophrenia were selected from the genome-wide association study (GWAS) of schizophrenia. (See, Schizophrenia Working Group of the Psychiatric Genomics Consortium, Nature, 511(7510):421-427, 2014). GWAS summary statistics were downloaded from the Psychiatric Genomics Consortium (PGC) website (med.unc.edu/pgc/results-and-downloads). SNVs with association P-value≤5×10⁻² were selected and merged with the SNVs in the Affymetrix 6.0 Array. The intersection of this merger produced a list of 122,395 SNVs. From this list, 120,000 SNVs were used to form a 3-color, 200×200 pixel image: the red channel used the first 200×200 SNVs, the blue channel used the second 200×200 SNVs, and the green channel used the last 200×200 SNVs. The genotypes of the SNVs, i.e., AA, Aa, and aa, were converted to the values of 0, 154, and 254, respectively, and the SNVs from each individual formed an AIO. An AIO for a schizophrenia patient is shown in FIG. 3A and an AIO for a healthy subject is shown in FIG. 3B.

The AIOs were then analyzed with the Keras/TensorFlow software (tensorflow.org/) (Abadi et al., “TensorFlow: A System for Large-Scale Machine Learning,” USENIX Assoc., 12^{th} USENIX Symposium on Operating Systems Design and Implementation, OSI1, 16:265-283, 2016; Abadi et al., “TensorFlow: Large-Scale Machine Learning on Heterogeneous Distributed Systems,” arXiv:1603.04467 [cs.DC], 2016) using a CNN architecture (Chen et al., 3rd IAPR Asian Conference on Pattern Recognition (ACPR), pp. 695-699, 2015; Ciresan et al., 2011 International Conference on Document Analysis and Recognition, pp. 1135-1139, 2011) using Python programming language. In this analysis, the goal was to classify the two groups of subjects in the GSE71443 dataset were classified. The GSE71443 data was randomly split 65/35, with 65% of the data used in model training and 35% of the data used as testing samples. To overcome potential overfitting, both the L1/L2 regularizers and dropout techniques were included in the model. After 1,000 training Epochs, the model obtained an accuracy of 0 769±0.040 (mean±st dev.) and an area under the curve (AUC) of 0.850±0.011. FIGS. 3C and 3D show a typical run of this classification.

An exemplary Python script for this example, binary classification of schizophrenia and healthy controls with GWAS-identified SNVs, is as set forth in Scheme 1.

### Scheme I

#Binary classification of genetic images made from SNVs selected
#from LD pruned genotype data. This example uses the
#convolutional neural network design with the GSE71443 data set
#downloaded from GEO database
#(ncbi.nlm.nih.gov/geo/query/acc.cgi?acc=GSE71443).
import pandas as pd
import numpy as np
import os
import tensorflow as tf
from keras import backend as K
from keras.models import Model, model from_json
from keras.layers.convolutional import Conv3D, MaxPooling3D,
AveragePooling3D
from keras.layers.convolutional import Conv2D, MaxPooling2D,
pyplot.plot(fpr test, tpr test, label=‘SNV test (area={:.3f})’.format(auc_test))
pyplot.xlabel(‘False positive rate’)
pyplot.ylabel(‘True positive rate’)
pyplot. title (‘ROC curve’)
pyplot.legend(loc=‘lower right’)
#pyplot.show( ) #enable this if want to see the figure instead of saving to file
pyplot.savefig(‘GSE71443_200C3_v10.6.0.j_ROC_figure.png’)
now=datetime.datetime.now( )
print(“The run is done by: \n”, now.strftime(“% Y-% m-% d % H:% M”))

This example demonstrates that with the use of 120,000 SNVs selected by GWAS threshold, i.e., P<=5e−2, the two groups of subjects are accurately classified as with or without a diagnosis of schizophrenic. In the literature, although there are reports that use GWAS-identified SNVs to predict schizophrenia diagnosis, there are two distinct aspects that are different in those legacy studies from the AIO analysis method. First, the current state of the art method is to use GWAS summary statistics to calculate polygenic risk scores, and then use these scores as predictors to predict diagnosis and evaluate disease susceptibility risks. In the polygenic risk score method, the effects of individual SNVs are aggregated, and therefore cannot be followed. With the AIO analysis method described here, the effects of individual SNVs were integrated into a single AIO that not only considered effects of multiple SNVs collectively (this was analogues to polygenic risk score), but also kept the effects of individual SNVs identifiable. This latter capability enables discovery of which SNVs were most relevant to the trait of interest. Second, compared to regression-based methods that have a limitation on the number of terms included in the model, the AIO-based method described herein is able to simultaneously consider a large number of SNVs for both the effects of individual SNVs and the effects of interactions among multiple SNVs. Employing the CNN architecture added another advantage over legacy methods since the effects of individual SNVs and interactions were dynamic and adjustable.

The implication of this example is that the model built by AIO with SNVs can be used reliably to predict the diagnosis of schizophrenia when the genotype data of an individual are available. This model could be used for risk assessment and early diagnosis for those individuals with high risks to develop schizophrenia.

### Example 4: Multi-Category Classification with AIOs-Distinguishing Squamous Cell Lung Cancer and Adenocarcinoma from Normal Controls Using a 3-Color Coding Scheme

The GSE25016 dataset has 291 subjects, of them, 59 are healthy controls, 155 are squamous cell lung cancer samples, and 77 are adenocarcinoma samples. Raw data downloaded from the GEO website included intensity data and genotype data. The platform used for GSE25016 genotyping was Affymetrix® 6.0 Array, which has 900,660 SNVs. (Thermo Fisher Scientific, Santa Clara, Calif, US).

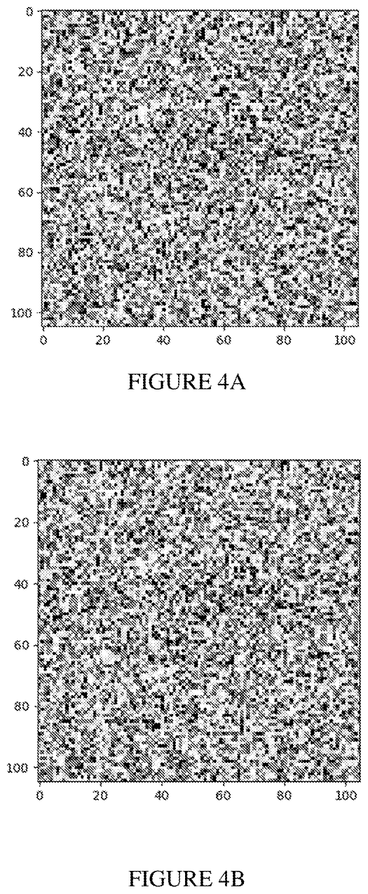

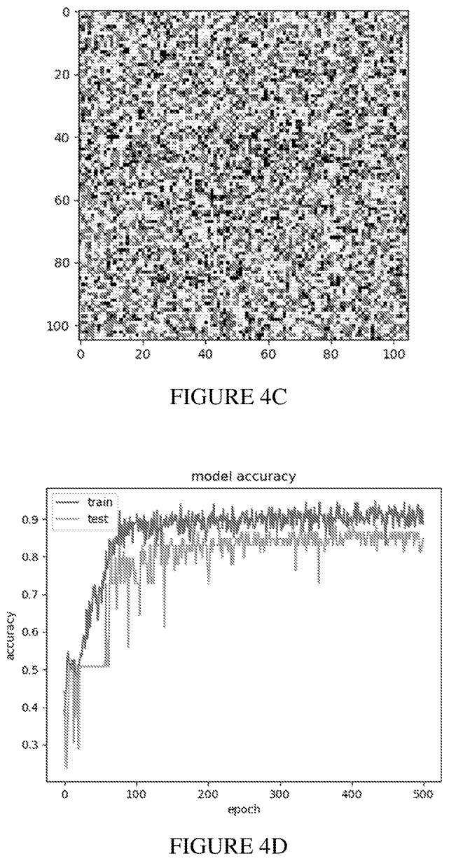

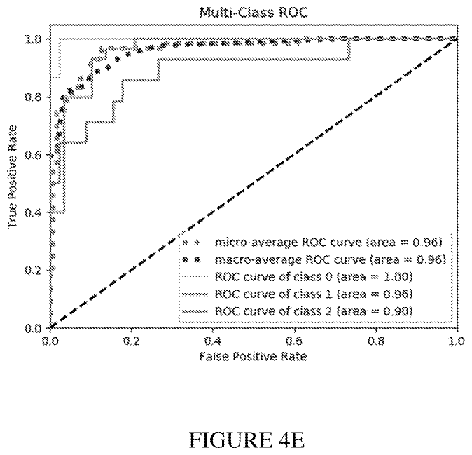

In this example, the objective was to classify the three groups of subjects included in the GSE25016 dataset, i.e., samples from healthy controls, samples from subjects with squamous cell lung cancer, and subjects with adenocarcinoma. Towards this goal, SNVs from an Affymetrix®6.0 Array were matched with an LD-pruned SNV list (r²=0.047) based on the 1000 Genome Project. This match produced a list of 37,768 SNVs. From these SNVs 33,075 SNVs were selected to make a 105×105×3 AIO for each subject for the samples in GSE25016 (FIGS. 4A, 4B, and 4C).

The AIOs were then analyzed with the Keras/TensorFlow software (tensorflow org/) (Abadi et al., 2016; Abadi et al., 2016) using a CNN architecture (Chen et al., 2015; Ciresan et al., 2011) using Python programming language. In this analysis, the goal was to classify the three groups of subjects in the GSE25016 dataset. The GSE25016 data was randomly split 80/20, with 80% of the data used in model training and 20% of the data used as testing/experimental samples. To overcome potential overfitting, both the L2 regularizer and dropout techniques were included in the model. After 500 training epochs, the model obtained an accuracy of 0.800±0.022, precision (true positive/[true positive+false positive]) of 0.811±0.027, and AUC of 0.946±0.101. Data obtained from a typical experiment is shown in FIG. 4D and FIG. 4E.

An exemplary Python script for the multi-category classification of lung cancer subtypes and healthy controls with LD pruned SNVs is provided in Scheme 2.

### Scheme 2

#Multi-category classification of lung cancer subtypes using
plt.plot(history.history[‘acc’])
plt.plot(history.history[‘val_ace’])
plt.plot(history.history[‘recall_m’])
plt.plot(history.history[‘val_recall_m’])
plt.plot(history.history[‘precision_m’])
plt.plot(history.history[‘val_precision_m’])
plt.title(‘model performance’)
plt.ylabel(‘accuracy/recall/precision’)
plt.xlabel(‘epoch’)
plt.legend([‘train ace’, ‘test ace’, ‘train recall’, ‘test recall’, ‘train precision’, ‘test precision’], loc=‘best’)
#pyplot.show( )
plt.savefig(‘GSE25016_105C3_v3.3.0.b_adam_train_test.png’)
now=datetime.datetime.now( )
print(“The run is done by: \n”, now.strftime(“% Y-% m-% d % H:% M”))

This example demonstrated that with the use of LD-pruned 33,075 SNVs selected from Affymetrix® SNP 6 array, the three groups of subjects characterized in the dataset are accurately classified based on the AIO images. The implication of this example is that the model built by AIO with SNVs can accurately estimates the probability of a subject with different lung cancer subtypes when his/her genotype data are available. This model therefore has utilities for subtype assessment and the prediction of treatment response.

### Example 5: Binary Classification with Whole Genome Transcription Data-Prediction of the Ki67 Status of Breast Cancer (BC)

This example employed the GSE81538 and GSE96058 datasets. (See, Bruefer et al., JCO Precision Oncology, 2.1-18, 2018) The GSE8I1538 dataset contains transcription and clinical data for 405 BC patients. The Ki67 antigen subtypes (Ki67⁺ and Ki67⁻) is one of the clinical data included in this dataset. Ki-67 is a cancer antigen found in growing, dividing cells but is absent in the resting phase of cell growth. Therefore, Ki67 is a good proliferation marker to follow the progress of BC tumors, and the Ki67 marker has been used to predict the aggressiveness and chemotherapy outcomes for BC. The GSE96058 dataset came from the same study as the GSE81538 dataset that contained similar clinical assessments as the GSE81538 dataset for 3,273 subjects with BC tumors. The GSE96058 dataset was an independent perspective study with median follow-up time of 52 months. In the present analyses, the GSE81538 was used as training data, and GSE96058 was used as validation data as described in the original publication (see reference above). Transcription and clinical data were downloaded from the NCBI GEO database. The transcription data contained the expression data of 18,802 genes.

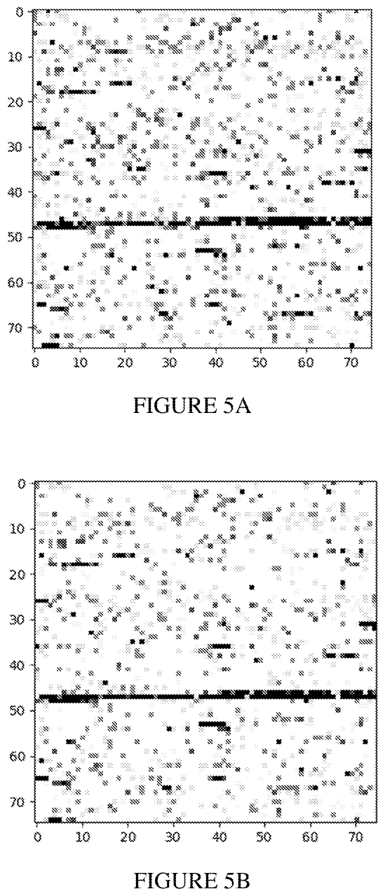

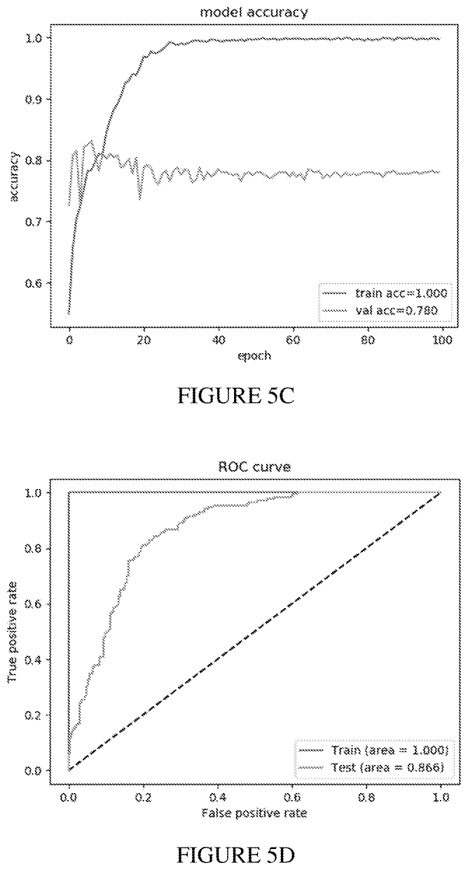

In this example, the first 16,875 genes of the shared 18,802 genes between the two datasets were employed. The expression data was rescaled to 0 to 254 gray-scale value, and arranged as an artificial image of 75×75×3 pixels, with the expression of each gene representing one pixel. This coding system is somewhat different than the genotype coding because the expression level of genes was continuous Therefore, the AIOs formed from these expression data had a full gray-scale, similar to a real black-white image. FIGS. 5A and 5B are representative of K167⁺ and Ki67⁻ subjects, respectively.

In this example, both convolutional and embedding layers were used to classify whether the samples were Ki67⁺ or Ki67⁻ using the TensorflowlKeras platform. The two convolutional layers used 256 neurons and were followed with 2 dense layers with 256 neurons. The embedding layer was followed by two dense layers with 256 neurons. The convolutional and embedding layers were concatenated together, and further followed with 4 dense layers (512 neurons). This neural network model accomplished an accuracy of 0.757+0.026 and AUC of 0.848+0.028. FIGS. 5C and 5D represent data from a typical run of this model. These results were about 10% better than the model reported in the original publication.

In this example, the concept of image coding described herein was extended to gene expression data, and the two subtypes of BC were successfully classified. Specifically, a set of 16,875 gene expression data was shown to be able to classify the Ki67⁺ and Ki67⁻ subtypes of BC. Based on the results learned from this example, this method of image recoding of expression data will work equally as well to classify other binary subtypes of BC and other diseases or biological conditions/traits.

### Example 6: Multi-Category Classification with Whole Genome Transcription Data-Prediction of BC PAM50 Subtypes

This example employed the GSE81538 and GSE96058 datasets. These datasets contain gene expression data obtained by the whole transcriptome sequencing method, and a set of clinical phenotypes. The PAM50 phenotype is one of the clinical data included in this data set PAM50 subtypes were initially classified by the use of a 50-gene signature, and the subtype assignment yielded a superior prognosis than classical immunohistochemistry factors (See, Parker et al., J. Clin. Oncol., 27(8):1160-1167, 2009). PAM50 has 4 subtypes (LumA, LumB, HER2-enriched, and Basal-like). In the GSE81538 dataset, there are 22 normal samples, 57 Basal-like tumors, 65 HER2-enriched tumors, 156 LumA tumors, and 105 LumB tumors. In the GSE96058 dataset, there are 202 normal samples, 325 Basal-like tumors, 307 HER2-enriched tumors, 1540 LumA tumors, and 695 LumB tumors. We used the GSE81538 dataset as training data, and the GSE96058 dataset as the validation dataset.

In this example, the same procedures used in Example 7 were used to select the 16,875 genes, and these genes were used to form the 75×75×3 pixel AIOs for the individuals in the GSE81538 and GSE96058 dataset in the AIO, each pixel represents the expression of a gene.

This example used both convolutional and embedding layers to construct the model to classify the BC subtypes and normal samples. The two convolutional layers had 128 neurons in each layer, followed with 3 dense layers (fully connected layers) with 128, 128, and 64 neurons, respectively. The embedding layer was followed with 3 dense layers (with 128, 128, and 64 neurons, respectively). The convolutional and embedding layers were concatenated together and followed by 3 dense layers (64, 64, and 32 neurons, respectively).

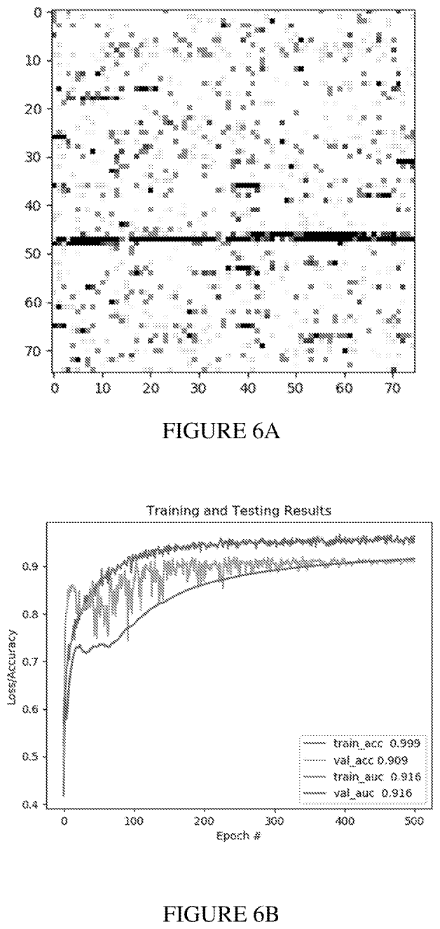

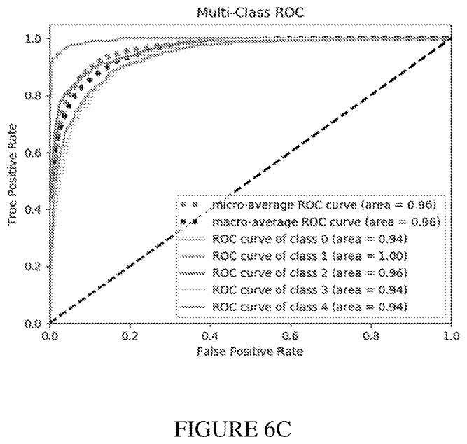

The model was trained for 500 epochs. The model achieved a classification accuracy of 0.93±0.01 and a micro-average AUC of 0.95±0.02. Data obtained from a typical training is shown in FIGS. 6A, 6B, and 6C. Compared to the original and other more recent reports, the image-based classification described in this method had equivalent or better performances. (See, Saal et al., Genom. Mol. Med., 7(1):20, 2015).

This example demonstrates that the methods described herein can classify multi-category data with expression data. Specifically, a set of 16,875 gene expression data is able to accurately classify the subtypes of PAM50 and healthy control samples.

The breadth and scope of the present application should not be limited by any of the above-described exemplary embodiments, but should be defined only in accordance with the following claims and their equivalents. That is, the above examples are included to demonstrate various exemplary embodiments of the described methods and systems. It will be appreciated by those of skill in the art that the techniques disclosed in the examples represent techniques discovered by the inventor to function well in the practice of the described methods and systems, and thus can be considered to constitute optional or exemplary modes for its practice. However, those of skill in the art will, in light of the present disclosure, appreciate that many changes can be made in these specific embodiments that are disclosed and still obtain a like or similar result without departing from the spirit and scope of the described methods and systems.

What is claimed is:

1. A computer-implemented method of identifying biological traits in subjects, the computer-implemented method comprising:
receiving, at a computing system, biological trait information of a subject and biological trait information of at least one control, wherein the biological trait information comprises discrete units of information, each discrete unit of information having at least one data type of specific variant information;
generating, by the computing system, artificial image objects, each artificial image object of the artificial image objects comprising an array of cells, each cell being a single unit addressable position within that artificial image object, wherein generating artificial image objects comprises:
for the biological trait information of the subject, assigning each discrete unit of information of the biological trait information of the subject to a cell of an artificial image object for the subject; and
for the biological trait information of each of the at least one control, assigning each discrete unit of information of the biological trait information of that control to a cell of an artificial image object for that control, wherein the artificial image object for each of the at least one control forms a training set of artificial image objects;
wherein each cell of the artificial image object for the subject and the artificial image object for each of the at least one control is accorded a specific graphic pixel signal corresponding to the at least one data type of specific variant information of the assigned discrete unit of information for that cell;
training, at the computing system, an artificial intelligence algorithm for classifying artificial image objects based on graphic pixel signals representing data types of specific variant information using the training set of artificial image objects; and
applying, at the computing system, the trained artificial intelligence algorithm to the artificial image object for the subject to determine a probability that a particular biological trait represented in the at least one control is present in the subject.

2. The computer-implemented method of claim 1, wherein each discrete unit of information is assigned to cells in no particular order or orientation other than being in a same addressable position across all artificial image objects including the artificial image object of the subject and the training set of artificial image objects.

3. The computer-implemented method of claim 1, wherein the cells of the artificial image object are addressable using x, y coordinates of an X vs Y axis.

4. The computer-implemented method of claim 1, wherein the at least one control comprises one or more positive controls, one or more negative controls, or a plurality of controls comprising one or more positive controls and one or more negative controls.

5. The computer-implemented method of claim 1, wherein each different possible value for a data type of specific variant information has a corresponding graphic pixel signal value.

6. The computer-implemented method of claim 5, wherein the corresponding graphic pixel signal value for that different possible value for the data type of specific variant information is a same value across all artificial image objects including the artificial image object of the subject and the training set of artificial image objects.

7. The computer-implemented method of claim 1, wherein the specific graphic pixel signal comprises intensity, shade, color, pattern, or combination thereof.

8. The computer-implemented method of claim 7, wherein each cell encodes at least two data types of specific variant information using a corresponding two specific graphic pixel signals selected from the intensity, shade, color, or pattern.

9. The computer-implemented method of claim 7, wherein each different possible value for a data type of specific variant information corresponds to a mutually distinguishable intensity, shade, color, or pattern.

10. The computer-implemented method of claim 1, wherein the biological trait information comprises genetic variant information.

11. The computer-implemented method of claim 10, wherein each discrete unit of information comprises two data types of specific variant information of a gene sequence and an expression level of the gene sequence.

12. The computer-implemented method of claim 10, wherein a number of cells of the array of cells of each artificial image object is 10 or more.

13. The computer-implemented method of claim 1, wherein the array of cells are in two dimensions, forming a two dimensional image object.

14. The computer-implemented method of claim 1, wherein the array of cells are in three dimensions, forming a three dimensional image object.

15. The computer-implemented method of claim 1, wherein the artificial intelligence algorithm is a machine learning (ML) algorithm.

16. The computer-implemented method of claim 1, wherein the artificial intelligence algorithm is an artificial neural network (ANN) selected from a convolutional neural network (CNN), a deep learning neural network (DNN), a deep, highly nonlinear neural network (NNN), a developmental network (DN), a long short-term memory network (LSTM), a recurrent neural network (RNN), a deep belief network (DBN), large memory storage and retrieval neural network (LAMSTAR), deep stacking network (DSN), spike-and-slab restricted Boltzmann machine network (ssRBM), or a multilayer kernel machine network (MKM).

17. The computer-implemented method of claim 1, wherein the particular biological trait represented in the at least one control is:
predisposition to one or more mental illnesses selected from the group consisting of: neurodevelopmental disorder, bipolar disorder, anxiety disorder, trauma related disorder, dissociative disorder, somatic symptom disorder, eating disorder, sleeping disorder, impulsive/disruptive/conduct disorder, addictive disorder, neurocognitive disorder, and personality disorder;
susceptibility to a cancer selected from one or more of a carcinoma, sarcoma, myeloma, leukemia, or lymphoma;
susceptibility to one or more cardiovascular or heart disease;
susceptibility to obesity; or
susceptibility to diabetes.

18. The computer-implemented method of claim 17, wherein:
the particular biological trait is predisposition to one or more mental illnesses, and wherein the method further comprises outputting, by the computing system, a recommendation for prescribing counseling to the subject and/or administering a pharmaceutically active agent to the subject that treats the mental illness when the particular biological trait is present in the subject, or
the particular biological trait is susceptibility to one or more indications including cancer, cardiovascular or heart disease, obesity, and diabetes, and wherein the method further comprises outputting, by the computing system, a recommendation for administering to the subject a pharmaceutically active agent that treats the one or more indications when the particular biological trait is present in the subject.

19. The computer-implemented method of claim 1, wherein the at least one data type of specific variant information of the biological trait information comprises genetic data, gene expression and/or function data, DNA methylation data, proteomic data, epigenomic data, metabolomic data, microbiomic data, or a combination thereof.

20. The computer-implemented method of claim 1, wherein the biological trait information comprises one or more protein expression level, one or more protein function data points, one or more post-translational modification variant data points, or a combination thereof.
